(19) United States
(12) Patent Application Publication
(10) Pub. No.: US 2009/0143283 A1
(43) Pub. Date: Jun. 4, 2009

(54) Pituitary Adenylate Cyclase Activating Peptide (PACAP) Receptor (VPAC2) Agonists and Their Pharmacological Methods of Use

(75) Inventors: Kevin Clairmont, Cheshire, CT (US); Kevin J. Lumb, Guilford, CT (US); Thomas Buckholz, Milford, CT (US); Arthur I. Salhanick, New Haven, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Pharmaceuticals Corporation, West Haven (US)

(21) Appl. No.: 10/586,124

(22) PCT Filed: Jan. 27, 2005
(86) PCT No.: PCT/US05/02609

§ 371 Date: Jun. 24, 2008

Related U.S. Application Data
(60) Provisional application No. 60/539,550, filed on Jan. 27, 2004.
(60) Provisional application No. 60/566,499, filed on Apr. 29, 2004.

(51) Int. Cl. A61K 38/16; C07K 14/00; C07K 16/00; A61P 3/10; G01N 33/53

(52) U.S. Cl. 514/12; 530/324; 530/389.1; 530/388.1; 435/7.1

(57) ABSTRACT

This invention provides novel peptides that function in vivo as agonists of the VPAC2 receptor. These insulin secretagogue polypeptides are shown to lower blood glucose in vivo more than controls upon glucose challenge. The polypeptides of this invention are also stable in formulation and have long half-lives. The peptides of the present invention provide a new therapy for patients with decreased endogenous insulin secretion, in particular type 2 diabetics. In particular, the invention is a polypeptide selected from a specific group of VPAC2-related polypeptides, or functional equivalents thereof. The invention is also directed to a method of treating a metabolic disease in a mammal comprising administering a therapeutically effective amount of the insulin secretagogue peptides to said mammal. Also disclosed are methods of making the peptides, both recombinant and synthetic.

This application claims benefit of U.S. Provisional Application Ser. No. 60/539,550, filed on Jan. 27, 2004 and U.S. Provisional Application Ser. No. 60/566,499, filed Apr. 29, 2004, the contents of which are incorporated herein by reference in their entirety.

### FIELD OF THE INVENTION

This invention relates to newly identified polypeptides and the use of such polypeptides for therapeutic purposes. More particularly, the polypeptides of the present invention are useful in stimulating the release of insulin from pancreatic β-cells in a glucose-dependent manner, thereby providing a treatment option for those individuals afflicted with metabolic disorders such as diabetes or impaired glucose tolerance, a prediabetic state.

### BACKGROUND OF THE INVENTION

Diabetes is characterized by impaired glucose metabolism manifesting itself, among other things, by an elevated blood glucose level in the diabetic patient. Underlying defects lead to a classification of diabetes into two major groups: type 1 diabetes, or insulin dependent diabetes mellitus (IDDM), which arises when patients lack β-cells producing insulin in their pancreatic islets of Langerhans; and type 2 diabetes, or non-insulin dependent diabetes mellitus (NIDDM), which occurs in patients with an impaired β-cell function and alterations in insulin action.

Type 1 diabetic patients are currently treated with insulin, while the majority of type 2 diabetic patients are treated with agents that stimulate β-cell function or with agents that enhance the tissue sensitivity of the patients towards insulin. Over time almost one-half of type 2 diabetic subjects lose their response to these agents and then must be placed on insulin therapy. The drugs presently used to treat type 2 diabetes are described below.

Alpha-glucosidase inhibitors (e.g., Precose® (Bayer Pharmaceuticals Corporation), Voglibose™ (Takeda Pharmaceuticals Company Limited), and Miglitol® (Bayer Pharmaceuticals Corporation)) reduce the excursion of postprandial glucose by delaying the absorption of glucose from the gut. These drugs are safe and provide treatment for mild to moderately affected diabetic subjects. However, gastrointestinal side effects have been reported in the literature.

Insulin sensitizers are drugs that enhance the body's response to insulin. Thiozolidinediones such as Avandia® (GlaxoSmithKline, rosiglitazone) and Actos™ (Takeda Pharmaceuticals Company Limited, pioglitazone) activate the peroxisome proliferator-activated receptor (PPAR) gamma subtype and modulate the activity of a set of genes that have not been well described. Rezulin™ (Warner-Lambert Company, troglitazone), the first drug in this class, was withdrawn because elevated liver enzyme levels and drug induced hepatotoxicity. These hepatic effects do not appear to be a significant problem in patients using Avandia® and Actos™. Even so, liver enzyme testing is recommended every 2 months in the first year of therapy and periodically thereafter. Avandia® and Actos™ seem to be associated with fluid retention and edema. Another potential side effect is weight gain. Avandia® is not indicated for use with insulin because of concern about congestive heart failure.

Insulin secretagogues (e.g., sulfonylureas (SFUs) and other agents that act by the ATP-dependent K+ channel) are another drug type presently used to treat type 2 diabetes. SFUs are standard therapy for type 2 diabetics that have mild to moderate fasting glycemia. The SFUs have limitations that include a potential for inducing hypoglycemia, weight gain, and high primary and secondary failure rates. Ten to 20% of initially treated patients fail to show a significant treatment effect (primary failure). Secondary failure is demonstrated by an additional 20-30% loss of treatment effect after six months on an SFU. Insulin treatment is required in 50% of the SFU responders after 5-7 years of therapy (Scheen, et al., Diabetes Res. Clin. Pract. 6:533-543, 1989).

Glucophage® (Lipha Corporation, metformin HCl) is a biguanide that lowers blood glucose by decreasing hepatic glucose output and increasing peripheral glucose uptake and utilization. The drug is effective at lowering blood glucose in mildly and moderately affected subjects and does not have the side effects of weight gain or the potential to induce hypoglycemia. However, Glucophage®) has a number of side effects including gastrointestinal disturbances and lactic acidosis. Glucophage®) is contraindicated in diabetics over the age of 70 and in subjects with impairment in renal or liver function. Finally, Glucophage® has primary and secondary failure rates similar to the SFUs.

Insulin treatment is instituted after diet, exercise, and oral medications have failed to adequately control blood glucose. This treatment has the drawbacks that it is an injectable, that it can produce hypoglycemia, and that it causes weight gain.

Because of the problems with current treatments, new therapies to treat type 2 diabetes are needed. In particular, new treatments to retain normal (glucose-dependent) insulin secretion are needed. Such new drugs should have the following characteristics: dependent on glucose for promoting insulin secretion (i.e., produce insulin secretion only in the presence of elevated blood glucose); low primary and secondary failure rates; and preserving islet cell function. The strategy to develop the new therapy disclosed herein is based on the cyclic adenosine monophosphate (cAMP) signaling mechanism and its effects on insulin secretion.

Cyclic AMP is a major regulator of the insulin secretion process. Elevation of this signaling molecule promotes the closure of the K+ channels following the activation of protein kinase A pathway. Closure of the K+ channels causes cell depolarization and subsequent opening of Ca⁺⁺ channels, which in turn leads to exocytosis of insulin granules. Little if any effects on insulin secretion occurs in the absence of low glucose concentrations (Weinhaus, et al., Diabetes 47:1426-1435, 1998). Secretagogues like PACAP (pituitary adenylate cyclase activating peptide), VIP (vasoactive intestinal peptide), and GLP-1 (glucagon-like peptide 1) use the cAMP system to regulate insulin secretion in a glucose-dependent fashion (Komatsu, et al., Diabetes 46:1928-1938, 1997; Filipsson, et al., Diabetes 50:1959-1969, 2001; Drucker, Endocrinology 142:521-527, 2001). Insulin secretagogues working through the elevation of cAMP such as GLP-1, VIP, and PACAP are also able to enhance insulin synthesis in addition to insulin release (Skoglund, et al., Diabetes 49:1156-1164, 2000; Borboni, et al., Endocrinology 140:5530-5537, 1999).

GLP-1 is released from the intestinal L-cell after a meal and functions as an incretin hormone (i.e., it potentiates glucose-induced insulin release from the pancreatic β-cell). It is a 37-amino acid peptide that is differentially expressed by the glucagon gene, depending upon tissue type. The clinical data that support the beneficial effect of raising CAMP levels in β-cells have been collected with GLP-1. Infusions of GLP-1 in poorly controlled type 2 diabetics normalized their fasting blood glucose levels (Gutniak, et al., New Eng. J. Med. 326:1316-1322, 1992) and with longer infusions improved the β-cell function to those of normal subjects (Rachman, et al., Diabetes 45:1524-1530, 1996). A recent report has shown that GLP-1 improves the ability of β-cells to respond to glucose in subjects with impaired glucose tolerance (Byrne, et al., Diabetes 47:1259-1265, 1998). All of these effects, however, are short-lived because of the short half-life of the peptide.

Amylin Pharmaceuticals has conducted clinical trials with Exendin-4 (AC2993), a 39 amino acid peptide originally identified in Gila Monster. Amylin has reported that clinical studies demonstrated improved glycemic control in type 2 diabetic patients treated with Exendin-4. However, the incidence of nausea and vomiting was significant.

PACAP is a potent stimulator of glucose-dependent insulin secretion from pancreatic β-cells. Three different PACAP receptor types (PAC1, VPAC1, and VPAC2) have been described (Harmar, et al., Pharmacol. Reviews 50:265-270, 1998; Vaudry, et al., Pharmacol. Reviews 52:269-324, 2000). PACAP displays no receptor selectivities, having comparable activities and potencies at all three receptors. PAC1 is located predominately in the CNS, whereas VPAC1 and VPAC2 are more widely distributed. VPAC1 is located in the CNS as well as in liver, lungs, and intestine. VPAC2 is located in the CNS, pancreas, skeletal muscle, heart, kidney, adipose tissue, testis, and stomach. Recent work argues that VPAC2 is responsible for the insulin secretion from β-cells (Inagaki, et al., Proc. Natl. Acad. Sci. USA 91:2679-2683, 1994; Tsutsumi, et al., Diabetes 51:1453-1460, 2002). This insulinotropic action of PACAP is mediated by the GTP binding protein Gs. Accumulation of intracellular cAMP in turn activates the nonselective cation channels in β-cells increasing [Ca++], and promotes exocytosis of insulin-containing secretory granules.

PACAP is the newest member of the superfamily of metabolic, neuroendocrine, and neurotransmitter peptide hormones that exert their action through the cAMP-mediated signal transduction pathway (Arimura, Regul. Peptides 37:287-303, 1992). The biologically active peptides are released from the biosynthetic precursor in two molecular forms, either as a 38-amino acid peptide (PACAP-38) and/or as a 27-amino acid peptide (PACAP-27) with an amidated carboxyl termini (Arimura, supra).

The highest concentrations of the two forms of the peptide are found in the brain and testis (Arimura, supra). The shorter form of the peptide, PACAP-27, shows 68% structural homology to vasoactive intestinal polypeptide (VIP). However, the distribution of PACAP and VIP in the central nervous system suggests that these structurally related peptides have distinct neurotransmitter functions (Koves, et al., Neuroendocrinology 54:159-169, 1991).

Recent studies have demonstrated diverse biological effects of PACAP-38, from a role in reproduction (McArdle, Endocrinology 135:815-817, 1994) to an ability to stimulate insulin secretion (Yada, et al., J. Biol. Chem. 269:1290-1293, 1994). In addition, PACAP appears to play a role in hormonal regulation of lipid and carbohydrate metabolism (Gray, et al., Mol. Endrocrinol. 15:1739-47, 2001); circadian function (Harmar, et al., Cell 109: 497-508, 2002); and the immune system, growth, energy homeostasis, and male reproductive function (Asnicar, et al., Endrocrinol. 143:3994-4006, 2002); regulation of appetite (Tachibana, et al., Neurosci. Lett. 339:203-206, 2003); as well as acute and chronic inflammatory diseases, septic shock, and autoimmune diseases (e.g., systemic lupus erythematosus) (Pozo, Trends Mol. Med. 9:211-217, 2003).

Vasoactive intestinal peptide (VIP) is a 28 amino acid peptide that was first isolated from hog upper small intestine (Said and Mutt, Science 169:1217-1218, 1970; U.S. Pat. No. 3,879,371). This peptide belongs to a family of structurally-related, small polypeptides that includes helodermin, secretin, the somatostatins, and glucagon. The biological effects of VIP are mediated by the activation of membrane-bound receptor proteins that are coupled to the intracellular cAMP signaling system. These receptors were originally known as VIP-R1 and VIP-R2, however, they were later found to be the same receptors as VPAC1 and VPAC2. VIP displays comparable activities and potencies at VPAC1 and VPAC2.

To improve the stability of VIP in human lung fluid, Bolin, et al., (Biopolymers 37:57-66, 1995) made a series of VIP variants designed to enhance the helical propensity of this peptide and reduce proteolytic degradation. Substitutions were focused on positions 8, 12, 17, and 25-28, which were implicated to be unimportant for receptor binding. Moreover, the ‘GGT’ sequence was tagged onto the C-terminus of VIP muteins with the hope of more effectively capping the helix. Finally, to further stabilize the helix, several cyclic variants were synthesized (U.S. Pat. No. 5,677,419). Although these efforts were not directed toward receptor selectivity, they yielded two analogs that have greater VPAC2 selectivity (Gourlet, et al., Peptides 18:403-408, 1997; Xia, et al., J. Pharmacol. Exp. Ther., 281:629-633, 1997).

There exists a need for improved peptides that have the glucose-dependent insulin secretagogue activity of PACAP, GLP-1, or Exendin-4, but with fewer side-effects, and preferably which are stable in formulation and have long plasma half-lives in vivo. Such improved in vivo half-life results from peptides with both decreased clearance and decreased susceptability to proteolysis. Furthermore, tighter control of plasma glucose levels may prevent long-term diabetic complications. Thus, new diabetic drugs should provide an improved quality of life for patients.

### SUMMARY OF THE INVENTION

This invention provides novel polypeptides that function in vivo as agonists of the VPAC2 receptor (hereafter, VPAC2) and are effective in the treatment of diseases and conditions that can be ameliorated by agents having VPAC2 agonist activity. Preferably, the polypeptides of this invention are selective VPAC2 agonists, having greater potency at VPAC2 than at VPAC1 and PAC1. For example, but not by way of limitation, these polypeptides stimulate insulin synthesis and release from pancreatic β-cells in a glucose-dependent fashion and subsequent plasma glucose reduction. These secretagogue polypeptides are shown to lower blood glucose in vivo more than vehicle control upon glucose challenge. Furthermore, the polypeptides of this invention are stable in formulation and have long plasma half-lives and long duration of action in vivo when derivatized.

The polypeptides of the present invention have improved stability to proteolysis by dipeptidylpeptidase IV (DPP4) and in plasma as compared to PACAP or VIP. While both VIP and PACAP27 have been reported to be resistant to cleavage by DPP4 (Zhu, et al., J. Biol. Chem. 278: 22418-22423, 2003), FIG. 2a demonstrates that these peptides are cleaved at longer time points, while the peptides of the present invention are resistant to cleavage at the time points tested. The derivatives of the present invention demonstrate an extended duration of action in vivo, supporting a dosing interval of less than once per day and, once per week or greater when derivatized.

The polypeptides of the present invention provide a therapy for patients with, for example, metabolic disorders such as those resulting from decreased endogenous insulin secretion, for example, type 2 diabetics, or for patients with impaired glucose tolerance, a prediabetic state that has a mild alteration in insulin secretion. In addition, the polypeptides of the present invention may be useful in the prevention and/or treatment of gestational diabetes, maturity-onset diabetes of the young (MODY), latent autoimmune diabetes adult (LADA), and associated diabetic dyslipidemia and other diabetic complications, as well as hyperglycemia, hyperinsulinemia, impaired glucose tolerance, impaired fasting glucose, dyslipidemia, hypertriglyceridemia, Syndrome X, and insulin resistance.

The polypeptides of the present invention may also be utilized in the prevention and/or treatment of obesity (e.g., regulation of appetite and food intake), atherosclerotic disease, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease; and for the prevention and/or treatment of lupus, polycystic ovary syndrome, carcinogenesis, and hyperplasia, asthma, male reproduction problems, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, septic shock, and other conditions identified herein, or function otherwise as described later herein.

One aspect of the invention is a polypeptide selected from the group consisting of SEQ ID NOs: 1 through 148, and fragments, derivatives, and variants thereof that demonstrate at least one biological function that is substantially the same as the polypeptides of the listed SEQ ID NOs. (collectively, “polypeptides of this invention”), including functional equivalents thereof. A further embodiment of this invention is a polypeptide selected from the group consisting of SEQ ID NOs: 1 through 37 and SEQ ID NOs: 112 through 148, and fragments, derivatives, and variants thereof that demonstrate at least one biological function that is substantially the same as the polypeptides of the listed SEQ ID NOs. Another embodiment of this invention is a polypeptide selected from the group consisting of SEQ ID NOs: 1 through 5 and SEQ ID NOs: 112 through 115, and fragments, derivatives and variants thereof that demonstrate at least one biological function that is substantially the same as the polypeptides of the listed SEQ ID NOs. A further embodiment of this invention is a polypeptide selected from the group consisting of SEQ ID NOs: 1 and 112, and fragments, derivatives and variants thereof that demonstrate at least one biological function that is substantially the same as the polypeptides of the listed SEQ ID NOs.

Antibodies and antibody fragments that selectively bind the polypeptides of this invention are also provided. Such antibodies are useful in detecting the polypeptides of this invention, and can be identified and made by procedures well known in the art. A polyclonal N-terminal IgG antibody and a monoclonal C-terminal Fab antibody have been generated which recognize polypeptides of this invention.

The invention is also directed to a method of treating diabetes, diabetes-related disorders, and/or other diseases or conditions affected by the polypeptides of this invention, for example, effected by the VPAC2 agonist function of the polypeptides of this invention, in a mammal, comprising administering a therapeutically effective amount of any of the polypeptides of the present invention or any polypeptide active at VPAC2 such as SEQ ID NOs: 1 through 148 to said mammal. Also disclosed are methods of making the polypeptides of this invention.

### BRIEF DESCRIPTION OF THE DRAWING

FIGS. 1a-1d depict amino acid sequences of polypeptides of SEQ ID NOs: 1 through 148. SEQ ID NOs: 112-148 refer to peptides that are PEGylated at the C-terminal cysteine via a maleimide linkage. The PEG may be of any length, for example, a 22 kD linear PEG or a 43 kD branched PEG or larger. FIG. 1e refers to related peptide standards.

FIGS. 2a and 2b depict stability of VPAC2 analogues to proteolytic cleavage by DPP4.

FIG. 3 depicts cAMP response of cells treated with VPAC peptides.

FIG. 4a 4b depict a sandwich ELISA assay for detection of peptides.

FIGS. 5a, 5b, and 5c demonstrate pharmacokinetic properties of VPAC peptides.

FIGS. 6a, 6b, 6c, and 6d illustrate in vivo efficacy.

### DETAILED DESCRIPTION OF THE INVENTION

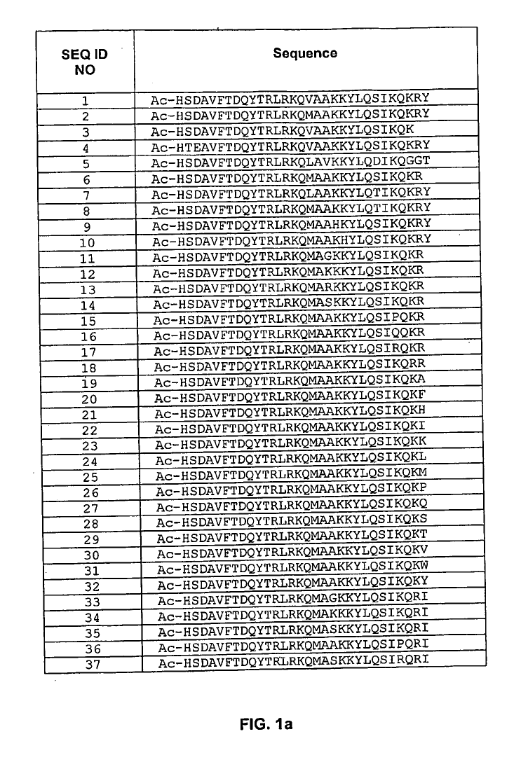

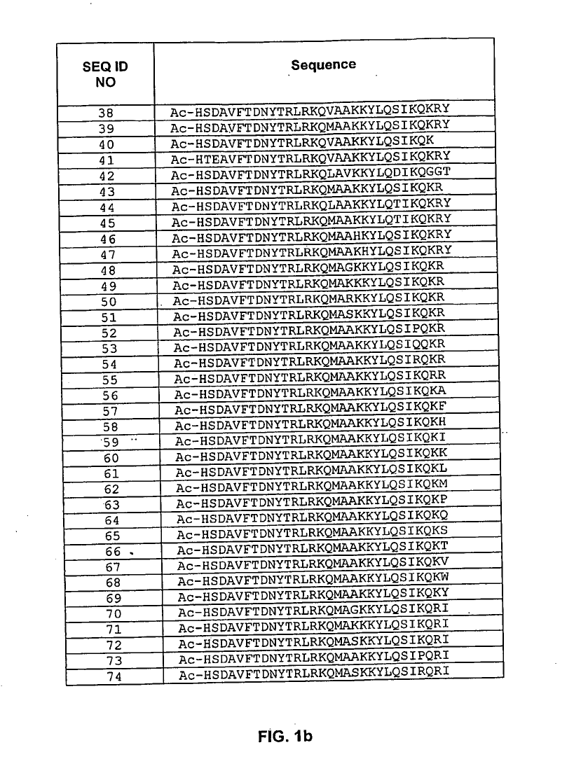

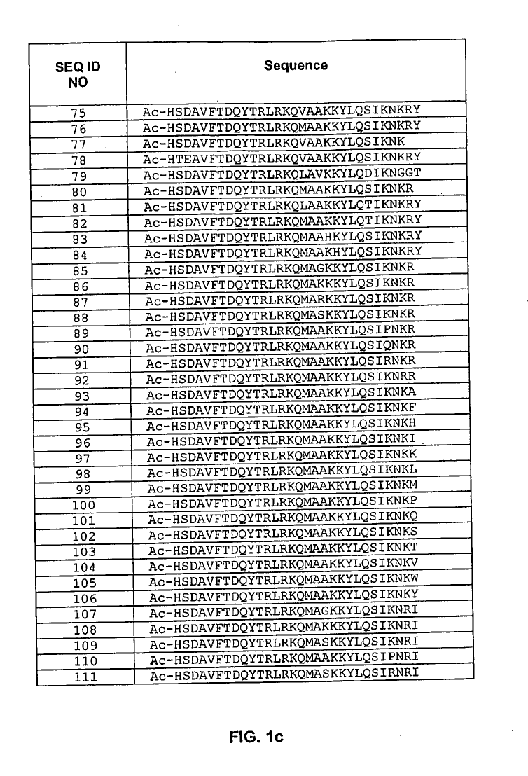

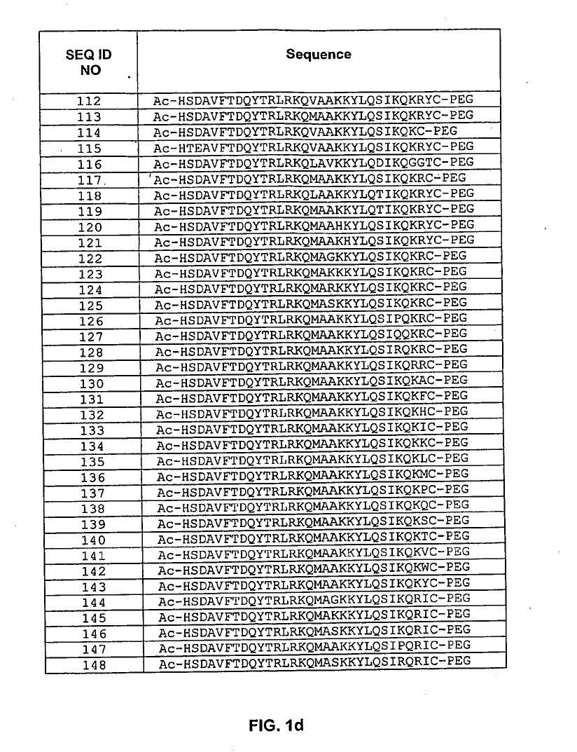

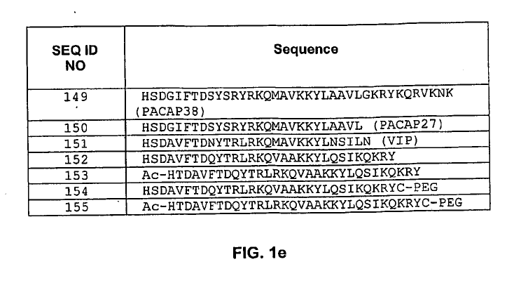

This invention provides novel polypeptides, and fragments, derivatives, and variants thereof that demonstrate at least one biological function that is substantially the same as the polypeptides of FIG. 1a-1d (collectively, polypeptides of this invention). The polypeptides of this invention function in vivo as VPAC2 agonists may be used for the prevention and/or treatment of such diseases or conditions as diabetes including type 2 diabetes, gestational diabetes, maturity-onset diabetes of the young (MODY) (Herman, et al., Diabetes 43:40, 1994); latent autoimmune diabetes adult (LADA) (Zimmet, et al., Diabetes Med. 11:299, 1994); and associated diabetic dyslipidemia and other diabetic complications, as well as hyperglycemia, hyperinsulinemia, impaired glucose tolerance, impaired fasting glucose, dyslipidemia, hypertriglyceridemia, Syndrome X, and insulin resistance.

In addition, the polypeptides of the present invention may also be utilized in the prevention and/or treatment of obesity (e.g., regulation of appetite and food intake), atherosclerotic disease, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease; and for the prevention and/or treatment of lupus, polycystic ovary syndrome, carcinogenesis, and hyperplasia, asthma, male reproduction problems including human sperm motility, ulcers, sleep disorders, and other conditions identified herein, or function otherwise as described later herein.

Furthermore, the polypeptides of this invention will stimulate insulin release from pancreatic β-cells in a glucose-dependent fashion. The polypeptides of this invention are also stable in both aqueous and non-aqueous formulations and exhibit a plasma half-life of greater than one hour, for example, demonstrating a plasma half-life greater than 6 hours.

The polypeptides of this invention are VPAC2 agonists. They are selective VPAC2 agonists with at least, for example, 10-fold selectivity for VPAC2 over VPAC1 and/or PAC1. The polypeptides of this invention stimulate insulin release into plasma in a glucose-dependent fashion without inducing a stasis or increase in the level of plasma glucose that is counterproductive to the treatment of, for example, type 2 diabetes. Additionally, the polypeptides of this invention are selective agonists of the VPAC2 receptor, thereby causing, for example, an increase in insulin release into plasma, while being selective against other receptors that are responsible for such disagreeable or dangerous side effects as gastrointestinal water retention, and/or unwanted cardiovascular effects such as increased heart rate or blood pressure.

The polypeptides of this invention are also stable in aqueous and non-aqueous formulations. For example, the polypeptides of this invention will exhibit less than 10% degradation at 37-40° C. over a period of one week, when dissolved in water (at pH between 7-8) or non-aqueous organic solvent. Furthermore, compositions and formulations of the present invention may comprise polypeptides of the present invention, and one or more pharmaceutically acceptable carriers, pharmaceutically acceptable diluents, and pharmaceutically acceptable solvents.

The polypeptides of this invention provide a therapy for patients with decreased endogenous insulin secretion or impaired glucose tolerance, for example, type 2 diabetes. That is, the polypeptides of the present invention are long-acting VPAC2 agonists that may be used to maintain, improve, and restore glucose-stimulated insulin secretion. Furthermore, a selective peptide agonist of the VPAC2 receptor will enhance glucose-dependent insulin secretion in the pancreas without causing the side effects associated with non-selective activation of the other PACAP receptors.

Certain terms used throughout this specification are defined below, and others will be defined as introduced. The single letter abbreviation for a particular amino acid, its corresponding amino acid, and three letter abbreviation are as follows: A, alanine (ala); C, cysteine (cys); D, aspartic acid (asp); E, glutamic acid (glu); F, phenylalanine (phe); G, glycine (gly); H, histidine (his); I, isoleucine (ile); K, lysine (lys); L, leucine (leu); M, methionine (met); N, asparagine (asn); P, proline (pro); Q, glutamine (gin); R, arginine (arg); S, serine (ser); T, threonine (thr); V, valine (val); W, tryptophan (trp); and Y, tyrosine (tyr).

“Functional equivalent” and “substantially the same biological function or activity” each means that degree of biological activity that is within about 30% to about 100% or more of that biological activity demonstrated by the polypeptide to which it is being compared when the biological activity of each polypeptide is determined by the same procedure. For example, a polypeptide that is functionally equivalent to a polypeptide of FIG. 1 is one that, when tested in the cyclic AMP (cAMP) scintillation proximity assay of Example 9, demonstrates accumulation of cAMP in CHO cell line expressing the human VPAC2 receptor.

The terms “fragment,” “derivative,” and “variant,” when referring to the polypeptides of FIG. 1, means fragments, derivatives, and variants of the polypeptides which retain substantially the same biological function or activity as such polypeptides, as described further below.

An analog includes a pro-polypeptide which includes within it, the amino acid sequence of the polypeptide of this invention. The active polypeptide of this invention can be cleaved from the additional amino acids that complete the pro-polypeptide molecule by natural, in vivo processes, or by procedures well known in the art such as by enzymatic or chemical cleavage. For example, the 28-amino acid native peptide VIP is naturally expressed as a much larger polypeptide which is then processed in vivo to release the 28-amino acid active mature peptide.

A fragment is a portion of the polypeptide which retains substantially similar functional activity, as described in the in vivo models disclosed herein.

A derivative includes all modifications to the polypeptide which substantially preserve the functions disclosed herein and include additional structure and attendant function (e.g., PEGylated or acetylated polypeptides which have greater half-life), fusion polypeptides which confer greater half-life, targeting specificity or an additional activity such as decreased toxicity to an intended target, as described further below.

The fragment, derivative, or variant of the polypeptides of the present invention may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the N-terminal acetyl group is replaced with another substituent on one or more of the first three amino acids or in which one or more of the first three amino acids is substituted with a conserved or non-conserved amino acid residue (preferably a conserved amino acid residue) and such substituted amino acid residue may or may not be one encoded by the genetic code in order to confer resistance to proteolysis, or (iv) one in which the mature polypeptide is fused with another compound, such as a compound to increase the half-life of the polypeptide (e.g., polyethyleneglycol or fatty acid), or (v) one in which the additional amino acids are fused to the mature polypeptide, such as a leader or secretory sequence or a sequence which is employed for purification of the mature polypeptide or a propolypeptide sequence, or (vi) one in which the polypeptide sequence is fused with a larger polypeptide (e.g., human albumin, an antibody or Fc, for increased duration of effect). Such fragments, derivatives, and variants and analogs are deemed to be within the scope of those skilled in the art from the teachings herein.

The derivatives of the present invention may contain conservative amino acid substitutions (defined further below) made at one or more predicted, preferably nonessential amino acid residues. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Non-conservative substitutions would not be made for conserved amino acid residues or for amino acid residues residing within a conserved protein domain, such as residues 19 and 27 where such residues are essential for protein activity such as VPAC2 activity and/or VPAC2 selectivity. Fragments, or biologically active portions include polypeptide fragments suitable for use as a medicament, to generate antibodies, as a research reagent, and the like. Fragments include peptides comprising amino acid sequences sufficiently similar to or derived from the amino acid sequences of a polypeptide of this invention and exhibiting at least one activity of that polypeptide, but which include fewer amino acids than the full-length polypeptides disclosed herein. Typically, biologically active portions comprise a domain or motif with at least one activity of the polypeptide. A biologically active portion of a polypeptide can be a peptide which is, for example, five or more amino acids in length. Such biologically active portions can be prepared synthetically or by recombinant techniques and can be evaluated for one or more of the functional activities of a polypeptide of this invention by means disclosed herein and/or well known in the art.

Variants of the polypeptides of this invention include polypeptides having an amino acid sequence sufficiently similar to the amino acid sequence of the SEQ ID NOs of FIG. 1 (a-d) or a domain thereof. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, about 75% through 98%, or identical are defined herein as sufficiently similar. For example, variants may be sufficiently similar to the amino acid sequence of the polypeptides of this invention.

Variants include polypeptides that differ in amino acid sequence due to mutagenesis. Variants that function as VPAC2 agonists can be identified by screening combinatorial libraries of mutants, for example, truncation mutants, of the polypeptides of this invention for VPAC2 agonist activity.

Moreover, derivatives of the present invention include mature polypeptides that have been fused with another compound, such as a compound to increase the half-life of the polypeptide and/or to reduce potential immunogenicity of the polypeptide (e.g., polyethylene glycol, “PEG” or fatty acid). In the case of PEGylation, the fusion of the polypeptide to PEG may be accomplished by any means known to one skilled in the art. For example, PEGylation may be accomplished by first introducing a cysteine mutation into the polypeptide to provide a linker upon which to attach the PEG, followed by site-specific derivatization with PEG-maleimide. As an example, the cysteine may be added to the C-terminus of the peptides (see, e.g., Tsutsumi, et al., Proc. Natl. Acad. Sci. USA 97(15):8548-53, 2000; Veronese, Biomaterials 22:405-417, 2001; Goodsoon & Katre, Bio/Technology 8:343-346, 1990). In addition to maleimide, numerous Cys reactive groups are known to those skilled in the art of protein crosslinking, such as the use of alkyl halides and vinyl sulfones (see, e.g., T. E. Creighton, Proteins, 2^{nd} Ed., 1993). In addition, the PEG may be introduced by direct attachment to the C-terminal carboxylate group, to the side chain of an C-terminal, to an internal amino acid such as Cys, Lys, Asp, or Glu, or to unnatural amino acids that contain similar reactive side chain moieties.

The linker between the PEG and the peptide crosslinking group may be varied. For example, the commercially available Cys-reactive 40 kDa PEG (mPEG2-MAL; Nektar, San Carlos, Calif.) employs a maleimide group for conjugation to Cys, and the maleimide group is attached to the PEG via a linker that contains a Lys. As a second example, the commercially available Cys-reactive 43 kDa PEG (GL2-400MA; NOF, Tokyo, Japan) employs a maleimide group for conjugation to Cys, and the maleimide group is attached to the PEG via a bisubstituted alkane linker.

The present invention exemplifies, but is not limited to, the use of Cys as a crosslinking site. It is well known that other moieties present in amino acids such as the N-terminal amino group, the C-terminal carboxylate, and the side chains of amino acids such as Lys, Arg, Asp, Glu, provide reactive groups that provide moieties suitable for covalent modification and attachment to PEG. Numerous examples of suitable crosslinking agents are known to those skilled in the art (see, e.g., T. E. Creighton, Proteins, 2^{nd} Ed., 1993). Such crosslinking agents may be linked to PEG as exemplified, but not limited to, by commercially available PEG derivatives containing amines, aldehydes, acetals, maleimide, succinimides, and thiols that are marketed, for example, by Nektar and NOF (e.g., Harris, et al., Clin. Pharmokinet. 40, 539-551, 2001).

The invention also provides chimeric or fusion polypeptides. The polypeptides of this invention can be composed of amino acids joined to each other by peptide bonds or modified peptide bonds (i.e., peptide isosteres), and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in research literature. Modifications may occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, e.g., Proteins, Structure and Molecular Properties. 2nd ed., T. E. Creighton, W. H. Freeman and Company, New York (1993); Posttranslational Covalent Modification of Proteins, B. C. Johnson, ed., Academic Press, New York, pgs. 1-12 (1983); Seifter, et al., Meth. Enzymol 182:626-646, 1990; Rattan, et al., Ann. N.Y. Acad. Sci. 663:4862, 1992).

The polypeptides of the present invention include the polypeptides of FIG. 1a-d (SEQ ID NOs: 1 through 148), as well as those sequences having insubstantial variations in sequence from them. An “insubstantial variation” would include any sequence addition, substitution, or deletion variant that maintains substantially at least one biological function of the polypeptides of this invention, for example, VPAC2 agonist activity, selective VPAC2 agonist activity, or insulin secreting activity demonstrated herein. These functional equivalents may include polypeptides which have at least about 90% identity, at least 95% identity, or at least 97% identity to the polypeptides of FIG. 1a-d, and also include portions of such polypeptides having substantially the same biological activity. However, any polypeptide having insubstantial variation in amino acid sequence from the polypeptides of FIG. 1a-d that demonstrates functional equivalency as described further herein is included in the description of the present invention.

As known in the art, “similarity” between two polypeptides may be determined by comparing the amino acid sequence and its conserved amino acid substitutes of one polypeptide to the sequence of a second polypeptide. Such conservative substitutions include those described above and by Dayhoff (The Atlas of Protein Sequence and Structure 5, 1978), and by Argos (EMBO J. 8:779-785, 1989). For example, amino acids belonging to one of the following groups represent conservative changes:

ala, pro, gly, gin, asn, ser, thr;

cys, ser, tyr, thr;

val, ile, leu, met, ala, phe;

lys, arg, his;

phe, tyr, trp, his; and

asp, glu.

The polypeptides of this invention may be a product of chemical synthetic procedures and as such, an isolated or purified polypeptide of this invention, or biologically active portion thereof, may be substantially free of chemical precursors or other chemicals when chemically synthesized. For example, an isolated polypeptide of this invention may have less than about 30% (by dry weight) of non-polypeptide, or contaminating, material. When a peptide of this invention is produced by chemical synthesis, the preparations may contain less than about 30% by dry weight of chemical precursors or non-invention chemicals.

The polypeptides of this invention may be conveniently isolated as described in the specific examples below. A preparation of purified polypeptide is, for example, at least about 70% pure; or about 85% through about 99% pure. Purity of the preparations can be assessed by any means known in the art, such as SDS-polyacrylamide gel electrophoresis and mass spectroscopy/liquid chromatography.

Also provided are related peptides and compounds (e.g., small molecule) within the understanding of those with skill in the art, such as chemical mimetics, organomimetics, or peptidomimetics. As used herein, the terms “mimetic,” “peptide mimetic,” “peptidomimetic,” “organomimetic,” and “chemical mimetic” are intended to encompass peptide derivatives, peptide analogs, and chemical compounds having an arrangement of atoms in a three-dimensional orientation that is equivalent to that of a peptide of the present invention. It will be understood that the phrase “equivalent to” as used herein is intended to encompass peptides or compounds having substitution(s) of certain atoms, or chemical moieties in said peptide, having bond lengths, bond angles, and arrangements in the mimetic compound that produce the same or sufficiently similar arrangement or orientation of said atoms and moieties to have the biological function of the peptides of the invention. In the peptide mimetics of the invention, the three-dimensional arrangement of the chemical constituents is structurally and/or functionally equivalent to the three-dimensional arrangement of the peptide backbone and component amino acid sidechains in the peptide, resulting in such peptido-, organo-, and chemical mimetics of the peptides of the invention having substantial biological activity. These terms are used according to the understanding in the art, as illustrated, for example, by Fauchere, (Adv. Drug Res. 15:29, 1986); Veber & Freidinger, (TINS p. 392, 1985); and Evans, et al., (J. Med. Chem. 30:1229, 1987), incorporated herein by reference.

It is understood that a pharmacophore exists for the biological activity of each peptide of the invention. A pharmacophore is understood in the art as comprising an idealized, three-dimensional definition of the structural requirements for biological activity. Peptido-, organo-, and chemical mimetics may be designed to fit each pharmacophore with current computer modeling software (computer aided drug design). Said mimetics may be produced by structure-function analysis, based on the positional information from the substituent atoms in the peptides of the invention.

Peptides as provided by the invention may be advantageously synthesized by any of the chemical synthesis techniques known in the art, particularly solid-phase synthesis techniques, for example, using commercially-available automated peptide synthesizers. The mimetics of the present invention may be synthesized by solid phase or solution phase methods conventionally used for the synthesis of peptides (see, e.g., Merrifield, J. Amer. Chem. Soc. 85:2149-54, 1963; Carpino, Acc. Chem. Res. 6:191-98, 1973; Birr, Aspects of the Merrifield Peptide Synthesis, Springer-Verlag: Heidelberg, 1978; The Peptides: Analysis, Synthesis, Biology, Vols. 1, 2, 3, and 5, (Gross & Meinhofer, eds.), Academic Press: New York, 1979; Stewart, et al., Solid Phase Peptide Synthesis, 2nd. ed., Pierce Chem. Co.: Rockford, Ill., 1984; Kent, Ann. Rev. Biochem. 57:957-89, 1988; and Gregg, et al., Int. J. Peptide Protein Res. 55:161-214, 1990, which are incorporated herein by reference in their entirety).

For example, solid phase methodology may be used. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods, or the like. Cycles of deprotection, neutralization, and coupling of successive protected amino acid derivatives are used to link the amino acids from the C-terminus according to the amino acid sequence. For some synthetic peptides, an FMOC strategy using an acid-sensitive resin may be used. Solid supports in this regard are divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenyl aminomethyl)-phenoxymethyl resin, 2,4-dimethoxybenzhydryl-amine resin, and 4-2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives are preferred: FMOC-Cys(Trit), FMOC-Ser(But), FMOC-Asn(Trit), FMOC-Leu, FMOC-Thr(Trit), FMOC-Val, FMOC-Gly, FMOC-Lys(Boc), FMOC-Gln(Trit), FMOC-Glu(OBut), FMOC-His(Trit), FMOC-Tyr(But), FMOC-Arg(PMC (2,2,5,7,8-pentamethylchroman-6-sulfonyl)), FMOC-Arg(BOC)₂, FMOC-Pro, and FMOC-Trp(BOC). The amino acid residues may be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexa-fluorophosphate), PyBOP (benzotriazole-1-yl-oxy-tris-pyrrolidinophosphonium hexafluoro-phosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. ActivaUon with HBTU (2-(1H-benzotriazole-1-yl), 1,1,3,3-tetramethyluronium hexafluorophosphate) or HATU (2-(1H-7-aza-benzotriazole-1-yl), 1,1,3,3-tetramethyluronium hexafluoro-phosphate) in the presence of HOBt or HOAt (7-azahydroxybenztriazole) is preferred.

The solid phase method may be carried out manually, although automated synthesis on a commercially available peptide synthesizer (e.g., Applied Biosystems 431A or the like; Applied Biosystems, Foster City, Calif.) may be used. In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (Applied Biosystems) may be used to generate the peptide sequence. Double and triple coupling, with capping by acetic anhydride, may also be used.

The synthetic mimetic peptide may be cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, may be used. The peptide may be separated from the resin by filtration and isolated by ether precipitation. Further purification may be achieved by conventional methods, such as gel filtration and reverse phase HPLC (high performance liquid chromatography). Synthetic mimetics according to the present invention may be in the form of pharmaceutically acceptable salts, especially base-addition salts including salts of organic bases and inorganic bases. The base-addition salts of the acidic amino acid residues may be prepared by treatment of the peptide with the appropriate base or inorganic base, according to procedures well known to those skilled in the art, or the desired salt may be obtained directly by lyophilization of the appropriate base.

Generally, those skilled in the art will recognize that peptides as described herein may be modified by a variety of chemical techniques to produce peptides having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric, and other organic salts, or may be converted to an amide. Thiols may be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue may be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives and analogs with the same or similar desired biological activity as the corresponding peptide but with more favorable activity than the peptide with respect to solubility, stability, and susceptibility to hydrolysis and proteolysis. Such derivatives and analogs include peptides modified at the N-terminal amino group, the C-terminal carboxyl group, and/or changing one or more of the amido linkages in the peptide to a non-amido linkage. It will be understood that two or more such modifications may be coupled in one peptide mimetic structure (e.g., modification at the C-terminal carboxyl group and inclusion of a —CH₂— carbamate linkage between two amino acids in the peptide).

Amino terminus modifications include alkylating, acetylating, adding a carbobenzoyl group, and forming a succinimide group. Specifically, the N-terminal amino group may be reacted to form an amide group of the formula RC(O)NH— where R is alkyl, for example, a lower alkyl, and is added by reaction with an acid halide, RC(O)Cl or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide an N-alkyl amide group of the formula RC(O)NR—. Alternatively, the amino terminus may be covalently linked to succinimide group by reaction with succinic anhydride. An approximately equimolar amount or an excess of succinic anhydride (e.g., about 5 equivalents) is used and the terminal amino group is converted to the succinimide by methods well known in the art including the use of an excess (e.g., 10 equivalents) of a tertiary amine such as diisopropylethylamine in a suitable inert solvent (e.g., dichloromethane), as described in Wollenberg, et al., (U.S. Pat. No. 4,612,132), and is incorporated herein by reference in its entirety. It will also be understood that the succinic group may be substituted with, for example, a C₂- through C₆-alkyl or —SR substituents, which are prepared in a conventional manner to provide for substituted succinimide at the N-terminus of the peptide. Such alkyl substituents may be prepared by reaction of a lower olefin (C₂- through C₆-alkyl) with maleic anhydride in the manner described by Wollenberg, et al., supra., and —SR substituents may be prepared by reaction of RSH with maleic anhydride where R is as defined above. In another advantageous embodiment, the amino terminus may be derivatized to form a benzyloxycarbonyl-NH— or a substituted benzyloxycarbonyl-NH— group. This derivative may be produced by reaction with approximately an equivalent amount or an excess of benzyloxycarbonyl chloride (CBZ-Cl), or a substituted CBZ-Cl in a suitable inert diluent (e.g., dichloromethane), for example, containing a tertiary amine to scavenge the acid generated during the reaction. In yet another derivative, the N-terminus comprises a sulfonamide group by reaction with an equivalent amount or an excess (e.g., 5 equivalents) of R—S(O)₂Cl in a suitable inert diluent (dichloromethane) to convert the terminal amine into a sulfonamide, where R is alkyl and preferably lower alkyl. For example, the inert diluent may contain excess tertiary amine (e.g., 10 equivalents) such as diisopropylethylamine, to scavenge the acid generated during reaction. Reaction conditions are otherwise conventional (e.g., room temperature for 30 minutes). Carbamate groups may be produced at the amino terminus by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate, where R is alkyl, preferably lower alkyl. For example, the inert diluent may contain an excess (e.g., about 10 equivalents) of a tertiary amine, such as diisopropylethylamine, to scavenge any acid generated during reaction. Reaction conditions are otherwise conventional (e.g., room temperature for 30 minutes). Urea groups may be formed at the amino terminus by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. For example, the inert diluent may contain an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group may be replaced by an ester (e.g., —C(O)OR where R is alkyl and preferably lower alkyl), resins used to prepare the peptide acids may be employed, and the side chain protected peptide may be cleaved with a base and the appropriate alcohol (e.g., methanol). Side chain protecting groups may be removed in the usual fashion by treatment with hydrogen fluoride to obtain the desired ester. In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide, and reaction with an alkylamine or a dialkylamine yields a side chain protected alkylamide or dialkylamide (i.e., the C-terminus is —C(O)NRR₁, where R and R₁ are alkyl and preferably lower alkyl). Side chain protection is then removed in the usual fashion by treatment with hydrogen fluoride to give the free amides, alkylamides, or dialkylamides.

In another alternative embodiment, the C-terminal carboxyl group or a C-terminal ester may be induced to cyclize by displacement of the —OH or the ester (—OR) of the carboxyl group or ester, respectively, with the N-terminal amino group to form a cyclic peptide. For example, after synthesis and cleavage to give the peptide acid, the free acid is converted in solution to an activated ester by an appropriate carboxyl group activator such as dicyclohexylcarbodiimide (DCC), for example, in methylene chloride (CH₂Cl₂), dimethyl formamide (DMF), or mixtures thereof. The cyclic peptide is then formed by displacement of the activated ester with the N-terminal amine. Cyclization, rather than polymerization, may be enhanced by use of very dilute solutions according to methods well known in the art.

Peptide mimetics as understood in the art and provided by the invention are structurally similar to the peptide of the invention, but have one or more peptide linkages optionally replaced by a linkage selected from the group consisting of: —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola, Chemistry and Biochemistry of Amino Acids, Peptides, and Proteins, (Weinstein, ed.), Marcel Dekker: New York, p. 267, 1983; Spatola, Peptide Backbone Modifications 1:3, 1983; Morley, Trends Pharm. Sci. pp. 463-468, 1980; Hudson, et al., Int. J. Pept. Prot. Res. 14:177-185, 1979; Spatola, et al., Life Sci. 38:1243-1249, 1986; Hann, J. Chem. Soc. Perkin Trans. I 307-314, 1982; Almquist, et al., J. Med. Chem. 23:1392-1398, 1980; Jennings-White, et al., Tetrahedron Lett. 23:2533, 1982; Szelke, et al., EP045665A; Holladay, et al., Tetrahedron Lett. 24:4401-4404, 1983; and Hruby, Life Sci. 31:189-199, 1982; each of which is incorporated herein by reference. Such peptide mimetics may have significant advantages over polypeptide embodiments, including, for example, more economical to produce, having greater chemical stability or enhanced pharmacological properties (such as half-life, absorption, potency, efficacy, etc.), reduced antigenicity, and other properties.

Mimetic analogs of the peptides of the invention may also be obtained using the principles of conventional or rational drug design (see, e.g., Andrews, et al., Proc. Alfred Benzon Symp. 28:145-165, 1990; McPherson, Eur. J. Biochem. 189:1-24, 1990; Hol, et al., in Molecular Recognition: Chemical and Biochemical Problems, (Roberts, ed.); Royal Society of Chemistry; pp. 84-93, 1989a; Hol, Arzneim-Forsch. 39:1016-1018, 1989b; Hol, Agnew Chem. Int. Ed. Engl. 25:767-778, 1986; the disclosures of which are herein incorporated by reference).

In accordance with the methods of conventional drug design, the desired mimetic molecules may be obtained by randomly testing molecules whose structures have an attribute in common with the structure of a “native” peptide. The quantitative contribution that results from a change in a particular group of a binding molecule may be determined by measuring the biological activity of the putative mimetic in comparison with the activity of the peptide. In one embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the peptides of the invention, as disclosed herein.

One method for performing rational mimetic design employs a computer system capable of forming a representation of the three-dimensional structure of the peptide, such as those exemplified by Hol, 1989a; Hol, 1989b; and Hol, 1986. Molecular structures of the peptido-, organo-, and chemical mimetics of the peptides of the invention may be produced using computer-assisted design programs commercially available in the art. Examples of such programs include SYBYL 6.5®, HQSAR™, and ALCHEMY 2000w (Tripos); GALAXY™ and AM2000% (AM Technologies, Inc., San Antonio, Tex.); CATALYSTS and CERIUS™ (Molecular Simulations, Inc., San Diego, Calif.); CACHE PRODUCTS™, TSAR™, AMBER™, and CHEM-X™ (Oxford Molecular Products, Oxford, Calif.) and CHEMBUILDER3D™ (Interactive Simulations, Inc., San Diego, Calif.).

The peptido-, organo-, and chemical mimetics produced using the peptides disclosed herein using, for example, art-recognized molecular modeling programs may be produced using conventional chemical synthetic techniques, and designed to accommodate high throughput screening, including combinatorial chemistry methods. Combinatorial methods useful in the production of the peptido-, organo-, and chemical mimetics of the invention include solid-phase synthesis and combinatorial chemistry arrays, as provided, for example, by SIDDCO (Tuscon, Ariz.); Tripos, Inc.; Calbiochem/Novabiochem (San Diego, Calif.); Symyx Technologies, Inc. (Santa Clara, Calif.); Medichem Research, Inc. (Lemont, Ill.); Pharm-Eco Laboratories, Inc. (Bethlehem, Pa.); or N.V. Organon (Oss, Netherlands). Combinatorial chemistry production of the peptido-, organo-, and chemical mimetics of the invention may be produced according to methods known in the art, including, but not limited to, techniques disclosed in Terrett, (Combinatorial Chemistry, Oxford University Press, London, 1998); Gallop, et al., J. Med. Chem. 37:1233-51, 1994; Gordon, et al., J. Med. Chem. 37:1385-1401, 1994; Look, et al., Bioorg. Med. Chem. Lett. 6:707-12, 1996; Ruhland, et al., J. Amer. Chem. Soc. 118:2534, 1996; Gordon, et al., Acc. Chem. Res. 29:144-54, 1996; Thompson & Ellman, Chem. Rev. 96:555-600, 1996; Fruchtel & Jung, Angew. Chem. Int. Ed. Engl. 35:17-42, 1996; Pavia, “The Chemical Generation of Molecular Diversity”, Network Science Center, www.netsci.org, 1995; Adnan, et al., “Solid Support Combinatorial Chemistry in Lead Discovery and SAR Optimization,” Id., 1995; Davies and Briant, “Combinatorial Chemistry Library Design using Pharmacophore Diversity,” Id., 1995; Pavia, “Chemically Generated Screening Libraries: Present and Future,” Id., 1996; and U.S. Pat. Nos. 5,880,972; 5,463,564; 5,331573; and 5,573,905.

The newly synthesized polypeptides may be substantially purified by preparative high performance liquid chromatography (see, e.g., Creighton, Proteins: Structures And Molecular Principles, WH Freeman and Co., New York, N.Y., 1983). The composition of a synthetic polypeptide of the present invention may be confirmed by amino acid analysis or sequencing by, for example, the Edman degradation procedure (Creighton, supra). Additionally, any portion of the amino acid sequence of the polypeptide may be altered during direct synthesis and/or combined using chemical methods with sequences from other proteins to produce a variant polypeptide or a fusion polypeptide.

Also included in this invention are antibodies and antibody fragments that selectively bind the polypeptides of this invention. Any type of antibody known in the art may be generated using methods well known in the art. For example, an antibody may be generated to bind specifically to an epitope of a polypeptide of this invention. “Antibody” as used herein includes intact Immunoglobulin molecules, as well as fragments thereof, such as Fab, F(ab′)₂, and Fv, which are capable of binding an epitope of a polypeptide of this invention. Typically, at least 6, 8, 10, or 12 contiguous amino acids are required to form an epitope. However, epitopes which involve non-contiguous amino acids may require more amino acids, for example, at least 15, 25, or 50 amino acids.

An antibody which specifically binds to an epitope of a polypeptide of this invention may be used therapeutically, as well as in immunochemical assays, such as Western blots, ELISAs, radioimmunoassays, immunohistochemical assays, immunoprecipitations, or other immunochemical assays known in the art. Various immunoassays may be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a polypeptide of this invention provides a detection signal at least 5-, 10-, or 20-fold higher than a detection signal provided with other proteins when used in an immunochemical assay. Preferably, antibodies which specifically bind to a polypeptide of this invention do not detect other proteins in immunochemical assays and can immunoprecipitate a polypeptide of this invention from solution.

Polypeptides of this invention, or fragments thereof, may be used to immunize a mammal, such as a mouse, rat, rabbit, guinea pig, monkey, or human, to produce polyclonal antibodies. If desired, a polypeptide of this invention or a fragment thereof may be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a polypeptide of this invention or a fragment thereof may be prepared using any technique which provides for the production of antibody molecules by continuous cell lines in culture. These techniques include, but are not limited to, the hybridoma technique, the human B cell hybridoma technique, and the EBV hybridoma technique (Kohler, et al., Nature 256:495-97, 1985; Kozbor, et al., J. Immunol. Methods 81:3142, 1985; Cote, et al., Proc. Natl. Acad. Sci. 80:2026-30, 1983; Cole, et al., Mol. Cell. Biol. 62:109-20, 1984).

In addition, techniques developed for the production of “chimeric antibodies,” the splicing of mouse antibody genes to human antibody genes to obtain a molecule with appropriate antigen specificity and biological activity, may be used (Morrison, et al., Proc. Natl. Acad. Sci. 81:6851-55, 1984; Neuberger, et al., Nature 312:604-08, 1984; Takeda, et al., Nature 314:452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences may be minimized by replacing residues which differ from those in the human sequences by site directed mutagenesis of individual residues or by grating of entire complementarity determining regions. Alternatively, humanized antibodies may be produced using recombinant methods (see, e.g., GB2188638B). Antibodies which specifically bind to a polypeptide of this invention may contain antigen binding sites which are either partially or fully humanized, as disclosed in U.S. Pat. No. 5,565,332.

Alternatively, techniques described for the production of single chain antibodies may be adapted using methods known in the art to produce single chain antibodies which specifically bind to a polypeptide of this invention. Antibodies with related specificity, but of distinct idiotypic composition, can be generated by chain shuffling from random combinatorial immunoglobin libraries (Burton, Proc. Natl. Acad. Sci. 88:11120-23, 1991).

Single-chain antibodies also may be constructed using a DNA amplification method, such as PCR, using hybridoma cDNA as a template (Thirion, et al., Eur. J. Cancer Prev. 5:507-11, 1996). Single-chain antibodies can be mono- or bi-specific, and can be bivalent or tetravalent. Construction of tetravalent, bi-specific single-chain antibodies is described, for example, in Coloma & Morrison (Nat. Biotechnol. 15:159-63, 1997). Construction of bivalent, bispecific single-chain antibodies is described in Mallender & Voss (J. Biol. Chem. 269:199-206, 1994).

A nucleotide sequence encoding a single-chain antibody may be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar, et al., Int. J. Cancer 61:497-501, 1995; Nicholls, et al., J. Immunol. Meth. 165:81-91, 1993).

Antibodies which specifically bind to a polypeptide of this invention may also be produced by inducing in vivo production in the lymphocyte population or by screening immunoglobulin libraries or panels of highly specific binding reagents as disclosed in the literature (Orlandi, et al., Proc. Natl. Acad. Sci. 86:38333-37, 1989; Winter, et al., Nature 349:293-99, 1991).

Other types of antibodies may be constructed and used therapeutically in methods of the invention. For example, chimeric antibodies may be constructed as disclosed in WO 93/03151. Binding proteins which are derived from immunoglobulins and which are multivalent and multi-specific, such as the “diabodies” also can be prepared (see, e.g., WO 94/13804).

Human antibodies with the ability to bind to the polypeptides of this invention may also be identified from the MorphoSys HuCAL® library as follows. A polypeptide of this invention may be coated on a microtiter plate and incubated with the MorphoSys HuCAL® Fab phage library. Those phage-linked Fabs not binding to the polypeptide of this invention can be washed away from the plate, leaving only phage which tightly bind to the polypeptide of this invention. The bound phage can be eluted, for example, by a change in pH or by elution with E. coli and amplified by infection of E. coli hosts. This panning process can be repeated once or twice to enrich for a population of antibodies that tightly bind to the polypeptide of this invention. The Fabs from the enriched pool are then expressed, purified, and screened in an ELISA assay.

Antibodies according to the invention may be purified by methods well known in the art. For example, antibodies may be affinity purified by passage over a column to which a polypeptide of this invention is bound. The bound antibodies can then be eluted from the column using a buffer with a high salt concentration.

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat a condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in a diabetic condition or disorder severity, while avoiding or minimizing adverse side effects associated with the given therapeutic treatment.

The term “pharmaceutically acceptable” means that the subject item is appropriate for use in a pharmaceutical product.

The polypeptides of the present invention, as a result of the ability to stimulate insulin secretion from pancreatic islet cells in vitro, and by causing a decrease in blood glucose in vivo, may be employed in treatment diabetes, including type 2 diabetes (non-insulin dependent diabetes mellitus). Such treatment may also delay the onset of diabetes and diabetic complications. The polypeptides may be used to prevent subjects with impaired glucose tolerance from proceeding to develop type 2 diabetes. Other diseases and conditions that may be treated or prevented using peptides of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); impaired glucose tolerance (IGT) (Expert Committee on Classification of Diabetes Mellitus, Diabetes Care 22 (Supp. 1):S5, 1999); impaired fasting glucose (IFG) (Charles, et al., Diabetes 40:796, 1991); gestational diabetes (Metzger, Diabetes, 40:197, 1991); and metabolic syndrome X.

The polypeptides of the present invention may also be effective in such disorders as obesity, and in the treatment of atherosclerotic disease, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease; and for the treatment of lupus, polycystic ovary syndrome, carcinogenesis, and hyperplasia, asthma, male reproduction problems, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, and septic shock.

The polypeptides of the present invention may also be useful for treating physiological disorders related to, for example, cell differentiation to produce lipid accumulating cells, regulation of insulin sensitivity and blood glucose levels, which are involved in, for example, abnormal pancreatic β-cell function, insulin secreting tumors and/or autoimmune hypoglycemia due to autoantibodies to insulin, autoantibodies to the insulin receptor, or autoantibodies that are stimulatory to pancreatic β-cells), macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

The polypeptides of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenyloin, thyroid hormone, β-adrenergic agents, α-interferon and drugs used to treat HIV infection.

In addition, the polypeptides of the invention may be used for treatment of asthma (Bolin, et al., Biopolymer 37:57-66, 1995; U.S. Pat. No. 5,677,419; showing that polypeptide R3P0 is active in relaxing guinea pig tracheal smooth muscle); hypotension induction (VIP induces hypotension, tachycardia, and facial flushing in asthmatic patients (Morice, et al., Peptides 7:279-280, 1986; Morice, et al., Lancet 2:1225-1227, 1983); male reproduction problems (Slow, et al., Arch. Androl. 43(1):67-71, 1999); as an anti-apoptosis/neuroprotective agent (Brenneman, et al., Ann. N.Y. Acad. Sci. 865:207-12, 1998); cardioprotection during ischemic events (Kalfin, et al., J. Pharmacol. Exp. Ther. 1268(2):952-8, 1994; Das, et al., Ann. N.Y. Acad. Sci. 865:297-308, 1998), manipulation of the circadian clock and its associated disorders (Hamar, et al., Cell 109:497-508, 2002; Shen, et al., Proc. Natl. Acad. Sci. 97:11575-80, 2000), and finally as an anti-ulcer agent (Tuncel, et al., Ann. N.Y. Acad. Sci. 865:309-22, 1998).

The polypeptides of the present invention may be used alone or in combination with additional therapies and/or compounds known to those skilled in the art in the treatment of diabetes and related disorders. Alternatively, the methods and polypeptides described herein may be used, partially or completely, in combination therapy.

The polypeptides of the invention may also be administered in combination with other known therapies for the treatment of diabetes, including PPAR agonists, sulfonylurea drugs, non-sulfonylurea secretagogues, α-glucosidase inhibitors, insulin sensitizers, insulin secretagogues, hepatic glucose output lowering compounds, insulin and anti-obesity drugs. Such therapies may be administered prior to, concurrently with or following administration of the polypeptides of the invention. Insulin includes both long and short acting forms and formulations of insulin. PPAR agonist may include agonists of any of the PPAR subunits or combinations thereof. For example, PPAR agonist may include agonists of PPAR-α, PPAR-γ, PPAR-δ or any combination of two or three of the subunits of PPAR. PPAR agonists include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a polypeptide of the invention include Precose®, Miglitol®, and Voglibose™. Insulin sensitizers that may be useful in treating diabetes include PPAR-γ agonists such as the glitazones (e.g., troglitazone, pioglitazone, englitazone, MCC-555, rosiglitazone, and the like); biguanides such as metformin and phenformin; protein tyrosine phosphatase-1B (PTP-1B) inhibitors; dipeptidyl peptidase IV (DP-IV) inhibitors; and thiazolidinediones and non-thiazolidinediones. Hepatic glucose output lowering compounds that may be useful in treating diabetes when administered with a polypeptide of the invention include metformin, such as Glucophage® and Glucophage XR®. Insulin secretagogues that may be useful in treating diabetes when administered with a polypeptide of the invention include sulfonylurea and non-sulfonylurea drugs: GLP-1, GIP, secretin, nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin. In one embodiment of the invention, polypeptides of the invention are used in combination with insulin secretagogues to increase the sensitivity of pancreatic β-3-cells to the insulin secretagogue.

Polypeptides of the invention may also be used in methods of the invention in combination with anti-obesity drugs. Anti-obesity drugs include β-3 agonists; CB-1 antagonists; neuropeptide Y inhibitors; appetite suppressants, such as, for example, sibutramine (Meridia®, Abbott Laboratories); and lipase inhibitors, such as, for example, orlistat (Xenical®, Roche Pharmaceuticals).

Polypeptides of the invention may also be used in methods of the invention in combination with drugs commonly used to treat lipid disorders in diabetic patients. Such drugs include, but are not limited to, HMG-CoA reductase inhibitors, nicotinic acid, lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Polypeptides of the invention may also be used in combination with anti-hypertensive drugs, such as, for example, β-blockers and ACE inhibitors. Examples of additional anti-hypertensive agents for use in combination with the polypeptides of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

Such co-therapies may be administered in any combination of two or more drugs (e.g., a polypeptide of the invention in combination with an insulin sensitizer and an anti-obesity drug). Such co-therapies may be administered in the form of pharmaceutical compositions, as described above.

Based on well known assays used to determine the efficacy for treatment of conditions identified above in mammals, and by comparison of these results with the results of known medicaments that are used to treat these conditions, the effective dosage of the polypeptides of this invention can readily be determined for treatment of each desired indication. The amount of the active ingredient (e.g., polypeptides) to be administered in the treatment of one of these conditions can vary widely according to such considerations as the particular polypeptide and dosage unit employed, the mode of administration, the period of treatment, the age and sex of the patient treated, and the nature and extent of the condition treated.

The total amount of the active ingredient to be administered may generally range from about 0.00001 mg/kg to about 1 mg/kg, and preferably from about 0.0001 mg/kg to about 0.1 mg/kg body weight per day. A unit dosage may contain from about 0.01 mg to about 20 mg of active ingredient, and may be administered one or more times per week. The weekly dosage for administration by injection, including intravenous, intramuscular, subcutaneous, and parenteral injections, and use of infusion techniques may be from about 0.0001 to about 0.1 mg/kg. The daily rectal dosage regimen may be from 0.001 to 1 mg/kg of total body weight. The transdermal concentration may be that required to maintain a daily dose of from 0.001 to 1 mg/kg.

Of course, the specific initial and continuing dosage regimen for each patient will vary according to the nature and severity of the condition as determined by the attending diagnostician, the activity of the specific polypeptide employed, the age of the patient, the diet of the patient, time of administration, route of administration, rate of excretion of the drug, drug combinations, and the like. The desired mode of treatment and number of doses of a polypeptide of the present invention may be ascertained by those skilled in the art using conventional treatment tests.

The polypeptides of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a therapeutically effective amount of a polypeptide. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a polypeptide is that amount which produces a result or exerts an influence on the particular condition being treated. The polypeptides described herein may be administered with a pharmaceutically-acceptable carrier using any effective conventional dosage unit forms, including, for example, immediate and timed release preparations, orally, parenterally, topically, or the like.

The polypeptides of this invention may be administered parenterally, that is, intravenously, intramuscularly, interperitoneally, or, preferably, subcutaneously, as injectable dosages of the polypeptide in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., polypeptide) with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the polypeptides of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patients ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The polypeptides described herein may be administered as the sole pharmaceutical agent or in combination with one or more other pharmaceutical agents where the combination causes no unacceptable adverse effects. For example, the polypeptides of this invention can be combined with known anti-obesity, or with known anti-diabetic or other indication agents, and the like, as well as with admixtures and combinations thereof.

The polypeptides described herein may also be utilized, in free base form or in compositions, in research and diagnostics, or as analytical reference standards, and the like. Therefore, the present invention includes compositions which are comprised of an inert carrier and an effective amount of a polypeptide identified by the methods described herein, or a salt or ester thereof. An inert carrier is any material which does not interact with the polypeptide to be carried and which lends support, means of conveyance, bulk, traceable material, and the like to the polypeptide to be carried. An effective amount of polypeptide is that amount which produces a result or exerts an influence on the particular procedure being performed.

Polypeptides are known to undergo hydrolysis, deamidation, oxidation, racemization and isomerization in aqueous and non-aqueous environment. Degradation such as hydrolysis, deamidation or oxidation can readily detected by capillary electrophoresis, mass spectrometry, or Edman degradation. Enzymatic degradation notwithstanding, polypeptides having a prolonged plasma half-life, or biological resident time, should, at minimum, be stable in aqueous solution. It is essential that polypeptide exhibits less than 10% degradation over a period of one day at body temperature. It is still more preferable that the polypeptide exhibits less than 5% degradation over a period of one day at body temperature. Because of the life time treatment in chronic diabetic patient, much preferably these therapeutic agents are convenient to administer, furthermore infrequently if by parenteral route. Stability (i.e., less than a few percent of degradation) over a period of weeks at body temperature will allow less frequent dosing. Stability (i.e., less than a few percent of degradation) to degradation through proteolysis, as to DPP4 or in plasma over periods of days to weeks will further support less frequent dosing. Stability in the magnitude of years at refrigeration temperature will allow the manufacturer to present a liquid formulation, thus avoid the inconvenience of reconstitution. Additionally, stability in organic solvent would provide polypeptide be formulated into novel dosage forms such as implant.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

It should be apparent to one of ordinary skill in the art that changes and modifications can be made to this invention without departing from the spirit or scope of the invention as it is set forth herein.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Peptide Synthesis Methodology

The following general procedure was followed to synthesize some of the polypeptides of the invention:

Peptide synthesis was carried out by the FMOC/t-Butyl strategy (Pennington & Dunn, Peptide Synthesis Protocols, Volume 35, 1994) under continuous flow conditions using Rapp-Polymere PEG-Polystyrene resins (Rapp-Polymere, Tubingen, Germany). At the completion of synthesis, peptides were cleaved from the resin and de-protected using TFA/DTT/H₂O/Triisopropyl silane (88/5/5/2). Peptides were precipitated from the cleavage cocktail using cold diethyl ether. The precipitate was washed three times with the cold ether, and then dissolved in 5% acetic acid prior to lyophilization. Peptides were checked by reversed phase chromatography on a YMC-Pack ODS-AQ column (YMC, Inc., Wilmington, N.C.) on a Waters ALLIANCE® system (Waters Corporation, Milford, Mass.) using water/acetonitrile with 3% TFA as a gradient from 0% to 100% acetonitrile, and by MALDI mass spectrometry on a VOYAGER DE™ MALDI Mass Spectrometer, (Model 5-2386-00, PerSeptive BioSystems, Framingham, Mass.). The peptide sample was added to the Matrix buffer (50/50 dH₂O/acetonitrile with 3% TFA) in a 1/1 ratio. Those peptides not meeting the purity criteria of >95% were purified by reversed phase chromatography on a Waters Delta Prep 4000 HPLC System (Waters Corporation, Milford, Mass.).

### Example 2

### Peptide Acetylation

Peptides were synthesized by standard methods well known in the art. Peptides were synthesiszed with an Applied Biosystems 430A peptide synthesizer using FMOC chemistry with HBTU activation on Rink amide resin and the N-terminus was acetylated with acetic anhydride. The peptide was cleaved with 84.6% TFA, 4.4% phenol, 4.4% water, 4.4% thioanisol, and 2.2% ethanedithiol. Peptides were precipitated from the cleavage cocktail using cold tertbutylmethyl ether. The precipitate was washed with the cold ether and dried under argon. Peptides were purified with reversed phase C18 chromatography with linear water/acetonitrile gradients containing 0.1% TFA. Peptide identity was confirmed with MALDI and electrospray mass spectrometry and with amino acid analysis.

### Example 3

### Peptide PEGylation

The half-life of a peptide in vivo may be increased through attachment of a polyethylene glycol (PEG) moiety to the peptide thereby reducing clearance of the peptide by the kidney and decreasing protease degradation of the peptide. The use of a VPAC2 receptor agonist peptide is severely limited by its very short half-life in vivo; however, attachment of a PEG moiety to the peptide (PEGylation) prolonged the half-life of the peptide sufficiently to allow for once/day to once/week treatment.

PEGylation may be performed by any method known to those skilled in the art. However, in this example, PEGylation was performed by introducing a unique cysteine mutation into the peptide followed by PEGylating the cysteine via a stable thioether linkage between the sulfhydryl of the peptide and maleimide group of the methoxy-PEG-maleimide reagent (Nektar (Inhale/Shearwater), San Carlos, Calif.; NOF, Tokyo, Japan). The unique cysteine may be introduced at the C-terminus of the peptide to minimize potential reduction of activity by PEGylation.

As an example, a 2-fold molar excess of mPEG-mal (MW 22 kD and 43 kD) reagent was added to 1 mg of peptide (e.g., SEQ ID NO:1 having a cysteine mutation at the C-terminus of the peptide) and dissolved in reaction buffer at pH 6 (0.1 M Na phosphate/0.1 M NaCl/0.1 M EDTA). After 30 minutes at room temperature, the reaction was terminated with 2-fold molar excess of DTT to mPEG-mal. The peptide-PEG-mal reaction mixture was applied to a cation exchange column to remove residual PEG reagents followed by gel filtration column to remove residual free peptide. The purity, mass, and number of PEGylated sites were determined by SDS-PAGE and MALDI-TOF mass spectrometry. When a 22 kD PEG was attached to peptides of the present invention, potent VPAC2 receptor activation was retained. Furthermore, VPAC2 versus VPAC1 and PAC1 selectivity of receptor activation was also retained. It is possible that PEGylation with a smaller PEG (e.g., a linear 22 kD PEG) may be less likely reduce activity of the peptide, whereas a larger PEG (e.g., a branched 43 kD PEG) will more likely reduce activity. However, the larger PEG will increase plasma half-life further so that once a week injection may be possible (Harris, et al., Clin. Pharmacokinet. 40:539-551, 2001).

### Example 4

### Pharmaceutical Composition—IV and SC Formulations

A sterile IV injectable formulation is prepared with 4 mg of a polypeptide of SEQ ID NO: 1, or a derivatized polypeptide having equivalent of 4 mg polypeptide content, and 1 L sterile saline, using any manufacturing process well known in the art. Higher concentrations of polypeptide may be used for SC formulation. In the case of the polypeptide identified as SEQ ID NO: 1, or a derivatized polypeptide, 4 mg is dissolved in 100 mL saline or DMSO and sterile vials after aseptic filtration, are filled with the composition.

### Example 5

### Mass Spectrometric Analysis of Peptides

Forty pmol/2 μl aliquots of peptides were diluted up to 10 μl with water. The HEPES buffer was removed by application of 50% of the sample (20 μmol/5 μl) to a conditioned Millipore C18 ZipTip, as per manufacturers instructions. Sample was eluted from the ZipTip with matrix (10 mg/ml alpha-cyanohydroxycinnamic acid in 50% ACN, 0.1% TFA) directly onto the MALDI plate. Samples were analyzed on an Applied Biosystems Voyager DE-PRO MALDI operated in the reflector ion mode. Data was collected in the 500-4000 Da range and resulting masses were compared to those expected by manual calculation.

### Example 6

### Edman Analysis of Peptides

Peptide samples were supplied for Edman degradation at 1 nmol/10 μl in 10 mM HEPES, pH 7.4, 5% TFA. Prior to Edman analysis, the HEPES buffer salt was removed by using an Applied Biosystems ProSorb sample cartridge as per manufacturers instructions. Briefly, sample was applied to a PVDF membrane and washed with 0.1% TFA, then the membrane was removed and inserted into the protein sequencer for Edman degradation. Edman degradation was carried out on an Applied Biosystems Procise 494HT protein sequencing system using the pulsed-liquid method according to manufacturer instructions. Sequence calls were made manually.

### Example 7

### Stability of Peptides

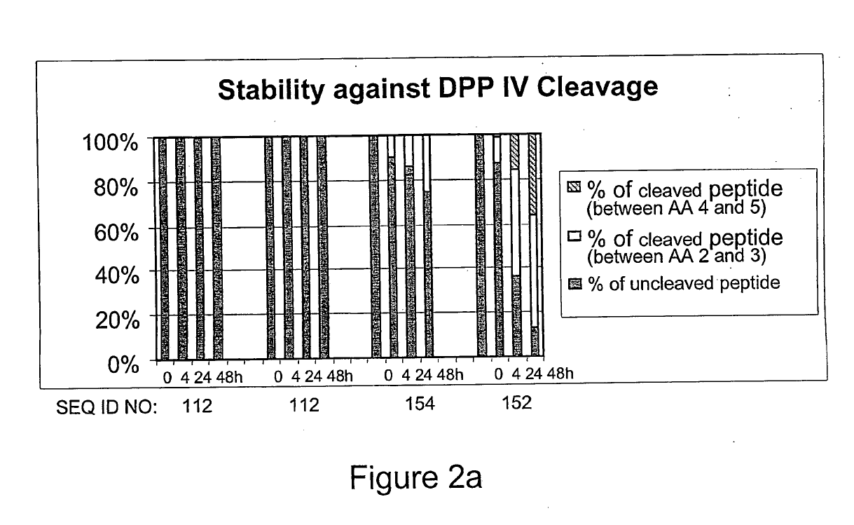

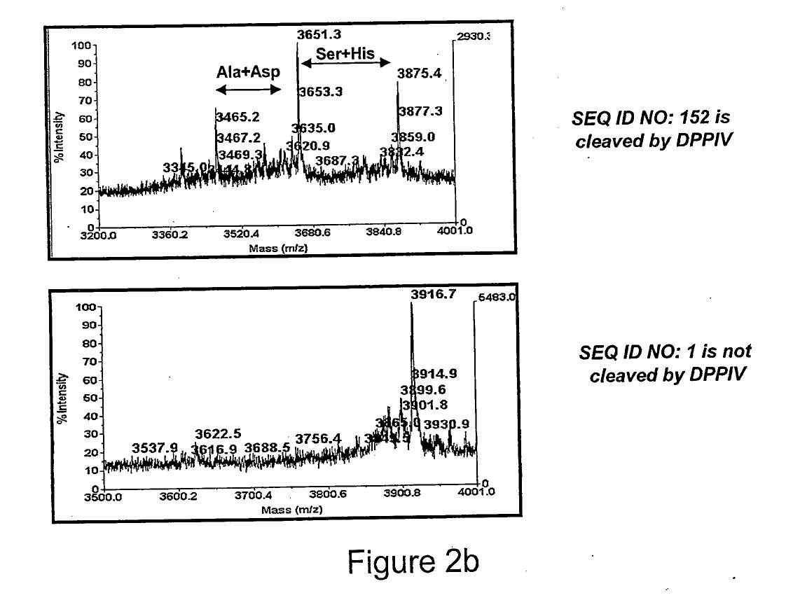

The formulations described in Example 4 were placed in constant stability chamber. Peptides were also analyzed for stability to degradation in solutions of DPP4 and in plasma. Samples were removed periodically for analysis by capillary electrophoresis, mass spectrometry, Edman degradation, ELISA, and assays of peptide activity, which are sensitive methods to detect degradation of polypeptide. The area of various peaks was summed and the area for peak of the parent polypeptide is divided by the total peak area. The quotient is the % purity. Since there are impurities present in the fresh polypeptide, the purity change is normalized by dividing the purity at different time point by the initial purity. For stability to DPP4 and plasma, peptides at 20 pmol/μl were incubated at 37° C. in the presence of 300 μM DPP-IV in 100 mM HEPES, pH 7.4 (FIG. 2a). At various timepoints, the reaction (2 μl aliquot) was terminated by addition of 1 μM DPP-IV inhibitor and freezing. For MALDI mass spectrometric analysis, T=0 hr, 1 hr, 5 hr, and 24 hr timepoints were evaluated. The results are plotted as percent intact peptide or peptide derivative as compared to degradation products.

### Example 8

### Binding of Peptides to PACAP1 and VPAC1 and 2 Receptors

CHO cells overexpressing the PAC1, VPAC1, and VPAC2 receptors were grown to confluency, scraped from their flasks and pelleted in a soft spin in 50 ml tubes. The pellets were resuspended in a Tris based homogenization buffer and homogenized in a Dounce tissue grinder with 30-40 manual strokes on ice. The suspension was spun in an ultracentrifuge which resulted in the pelleting of membranes. This pellet was resuspended in a small amount of homogenization buffer and a protein concentration was determined through the use of a BCA kit from Pierce.

A binding reaction containing 10 μg membrane protein, 0.1 nM ¹²⁵I-PACAP 27, and a dose curve of compound to be tested was incubated in a 96-well plate at 37° C. for 20 minutes. The reaction was stopped by placement of the plate on ice for 20 minutes. The reaction was added to a filter plate preincubated with 0.1% PEI to avoid non-specific binding, processed on a vacuum manifold and washed several times with a BSA based wash solution. The filter plate was dried, scintillant added, and read on a MicroBeta counter. The data was analyzed and presented in Prism graphs.

### Example 9

### Elevation of cAMP in Response to Peptides

CHO cells expressing the VPAC2 peptide were plated in 96-well plates at 8×10⁴ cells/well and grown at 37° C. for 24 hours in AMEM, nucleosides, glutamine (Gibco/BRL, Rockville, Md.), 5% FBS, 100 μg/mL Pen/Strep, 0.4 mg/mL hygromycin, and 1.5 mg/mL Geneticin (Gibco/BRL). The media was removed and the plates were washed with PBS. The cells were incubated with a peptide (in 10 mM HEPES, 150 mM NaCl, 5 mM KCl, 2.5 mM CaCl₂, 1.2 mM KH₂PO₄, 1.2 mM MgSO₄, 25 mM NaHCO₃ (pH 7.4) with 1% BSA and 100 μM IBMX) for 15 minutes at 37° C. Cyclic AMP in the cell extracts was quantitated using the cAMP SPA direct screening assay system (Amersham Pharmacia Biotech Inc., Piscataway, N.J.). The amount of cAMP present in the lysates was determined following instructions provided with this kit. The amount of cAMP (in pmol) produced at each concentration of peptide was plotted and analyzed by nonlinear regression using Prizm software to determine the EC₅₀ for each peptide.

Alternatively, the elevation of cAMP in response to receptor activation can be measured in a reporter cell line, such as CHO, which not only expresses the desired receptor but which also expresses a reporter, such as luciferase, linked to a cAMP response element (CRE). Such cell are plated in 96 well plates at 10⁴ cells per well and grown at 37° C. for 48 hours in aMEM, nucleosides, glutamine (Gibco/BRL, Rockville, Md.), 5% FBS, 100 μg/mL Pen/Strep, 0.4 mg/mL hygromycin, and 1.5 mg/mL Geneticin (Gibco/BRL). The cells are then incubated with peptide for 6 hours, the media removed, and Bright-Glo reagent (Promega) added. The signal is detected using a scintillation counter.

The polypeptides of this invention are designed based on VIP, which has been shown to lack activity at PAC1 (Vaudry, et al., Pharmacol. Rev. 52:269-324, 2000). Therefore, it is believed that the polypeptides of this invention do not possess appreciable activity at PAC1.

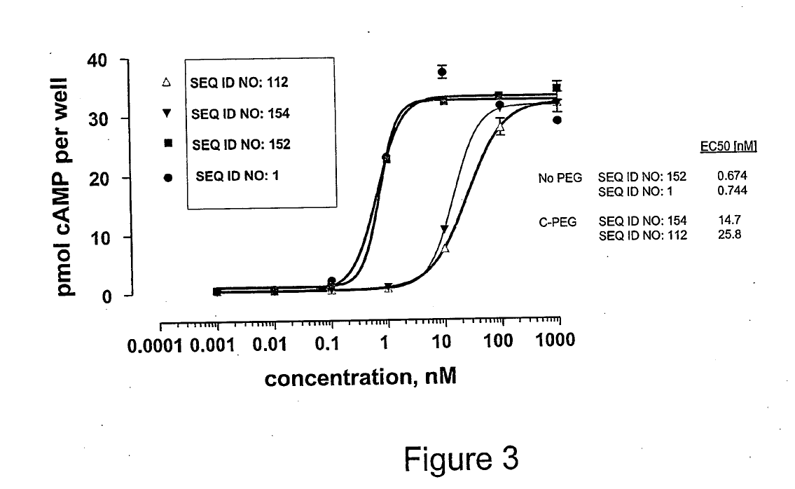

The results of this assay with representative polypeptides of this invention are shown in FIG. 3. Peptides identified as SEQ ID NOs: 1 and 112 are potent agonists of the VPAC2 receptor, activating the receptor to 100% the maximal level of receptor activation achieved by the endogenous peptide, PACAP-27. Furthermore, the peptides identified as SEQ ID Nos: 1, 112, 152, and 154 are selective VPAC2 receptor agonists, possessing very weak agonist activity on VPAC1. PACAP-27 is a potent agonist of VPAC1.

### Example 10

### Insulin Secretion from Dispersed Rat Islet Cells

Insulin secretion of dispersed rat islets mediated by a number of peptides of the present invention was measured as follows. Islets of Langerhans, isolated from SD rats (200-250 g), were digested using collagenase. The dispersed islet cells were treated with trypsin, seeded into 96 V-bottom plates, and pelleted. The cells were then cultured overnight in media with or without peptides of this invention. The media was aspirated, and the cells were pre-incubated with Krebs-Ringer-HEPES buffer containing 3 mM glucose for 30 minutes at 37° C. The pre-incubation buffer was removed, and the cells were incubated at 37° C. with Krebs-Ringer-HEPES buffer containing the appropriate glucose concentration (e.g., 8 mM) with or without peptides for an appropriate time. In some studies, an appropriate concentration of GLP-1 was also included. A portion of the supernatant was removed and its insulin content was measured by SPA. The results were expressed as “fold over control” (FOC).

In this assay, an increase of insulin secretion from dispersed rat islet cells was defined as an increase of at least 1.4-fold. The VPAC2 receptor agonist component of the polypeptides of this invention produced an increase in insulin secretion from dispersed islet cells by at least 1.4-fold to about 1.7-fold.

### Example 11

### Generation of Peptide Specific Antibodies and Peptide Measurement by ELISA

Polyclonal antibodies specific to the polypeptides of the present invention were generated by synthesizing a specific fragment of a polypeptide of this invention using an ABI 433A peptide synthesizer. The peptide was then cleaved from the resin, and purified on a Beckman System Gold Analytical and Preparative HPLC system. A Perspective MALDI mass spectrophotometer system was used to identify the correct product. The peptide was dried using a-lyophilizer. The peptide (2 mg) was then conjugated to keyhole limpet hemocyanin (KLH) via the free sulphydryl group on the Cys.

Female New Zealand White rabbits were immunized on Day 0, 14, 35, 56, and 77. On Day 0, each rabbit was injected subcutaneous with 250 μg peptide and complete Freund's adjuvant. Subsequent immunizations utilized 125 μg peptide per rabbit. Bleeds were started on Day 21 and continued at 21-day intervals thereafter. Purification of anti-peptide antibodies was performed by passing the crude serum over a specific peptide affinity purification column. The antibody titer was determined by ELISA.

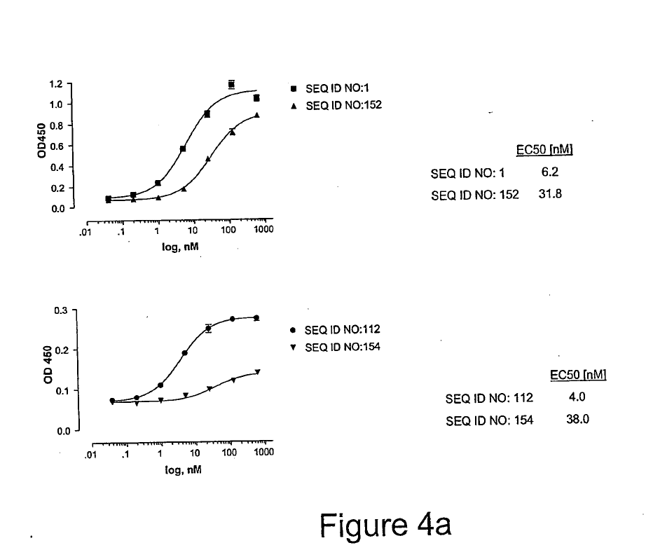

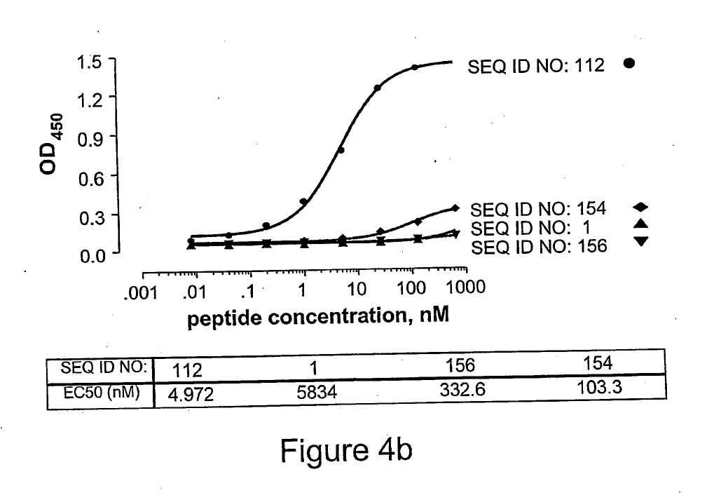

A 96-well immulon 4HBX plate was coated with a C-terminal Morphosys F(ab) antibody, specific to the peptides of the present invention, and allowed to incubate ovenight at 4° C. The plate was then blocked to prevent non-specific binding. Then, peptide standards (2500 ng/mL-160 pg/mL) were diluted in 33% plasma and the samples were diluted 1:3 in buffer followed by incubation for 1.5 hours at room temperature. After washing, a polyclonal N-terminal antibody specific to the peptides of this invention was incubated on the plate for one hour. This was followed by the addition of horseradish peroxidase (HRP)-donkey-anti-rabbit antibody and the samples and standards were incubated for another hour. Detection was assessed following incubation with 3,3′,5,5′-tetramethylbenzidine (TMB) solution, and the plate is read at OD₄₅₀ (FIG. 4a).

Alternatively, the 96-well immulon 4HBX plate was coated with a polyclonal N-terminal antibody, specific to the peptides of the present invention, and allowed to incubate overnight at 4° C. The plate was then blocked to prevent non-specific binding. Then, peptide standards (2500 ng/mL-160 pg/mL) were diluted in 50% plasma, and the samples were diluted 1:2 in buffer followed by incubation for 1.5 hours at room temperature. After washing, a monoclonal anti-PEG antibody specific to the peptides of this invention was incubated on the plate for one hour. This was followed by the addition of horseradish peroxidase (HRP)-anti-mouse antibody and the samples and standards were incubated for another hour. Detection was assessed following incubation with 3,3′,5,5′-etramethylbenzidine (TMB) solution, and the plate is read at OD₄₅₀ (FIG. 4b).

### Example 12

### Pharmacokinetics of Peptides Following IV and Subcutaneous Dosing

Plasma samples are transferred to a microcentrifuge tube and an equal volume of acetonitrile is added to the sample (a 50% final concentration). The sample is vigorously vortexed for about 5 minutes and allowed to sit on ice for 10 minutes. The sample is again vortexed for about 1 minute, and then centrifuged for 30 minutes in a microcentrifuge (4° C.) at maximum (about 15,000×g).

Following centrifugation, the aqueous phase is carefully transferred to a clean centrifuge tube, and the sample is centrifuged for 5 minutes in a microcentrifuge (4° C.) at maximum speed (about 15,000×g). The extracted sample is dried under vacuum using a Speed Vac SC110 (Savant) with a medium heat setting until dry. The sample is resuspended in an appropriate volume of sterile water and is maintained at 4° C. The sample is then sonicated in a sonic bath for 10 minutes at room temperature prior to analysis. The peptide concentration is determined utilizing the reporter assay as described in Example 9.

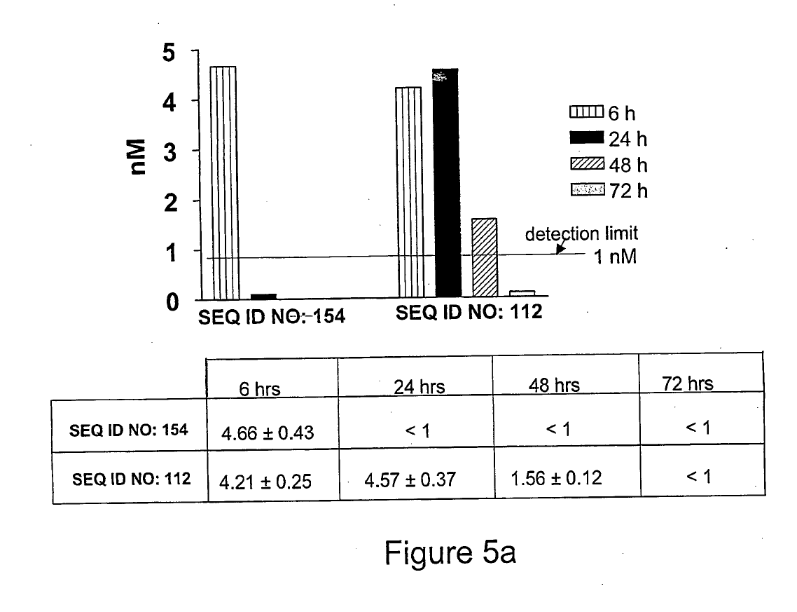

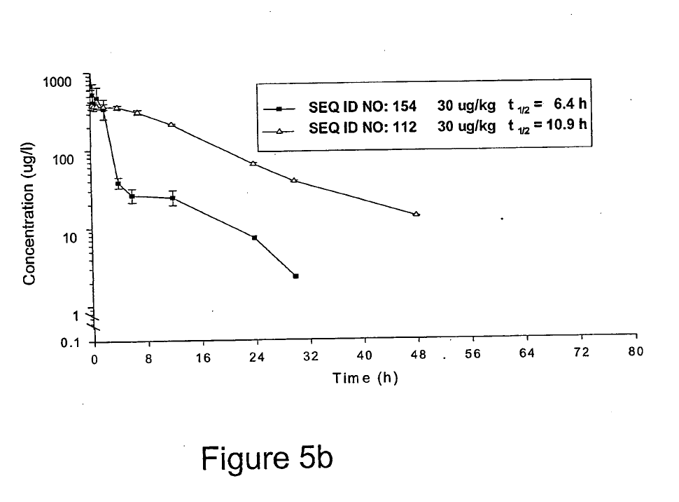

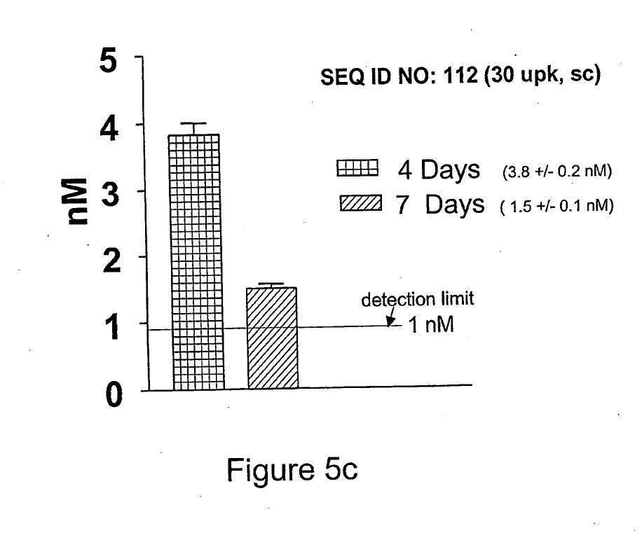

Using this method, the pharmacokinetic properties of SEQ ID NO: 112 were compared to that of SEQ ID NO: 154 (FIG. 5a). The peptide acetylation present on SEQ ID NO: 112 demonstrates improved pharmacokinetic properties, being detected at 24 and 48 hours, while the non-acetylated peptide, SEQ ID NO: 154, is only detected at 6 hours. Further studies comparing these two peptide demonstrated that SEQ ID NO: 112 had a half-life of 10.9 hours in the rat, while that of the non-acetylated SEQ ID NO: 154 was approximately 6 hours (FIG. 5b).

Plasma peptide concentrations may also be measured using ELISA, as described in Example 11. Using this methodology, the pharmacokinetics of SEQ ID NO: 112 was characterized further (FIG. 5c). In dog, SEQ ID NO: 112 remains present at detectable concentration for one week.

### Example 13

### Effect of PEGylated Peptides on Intraperitoneal Glucose Tolerance in Rats

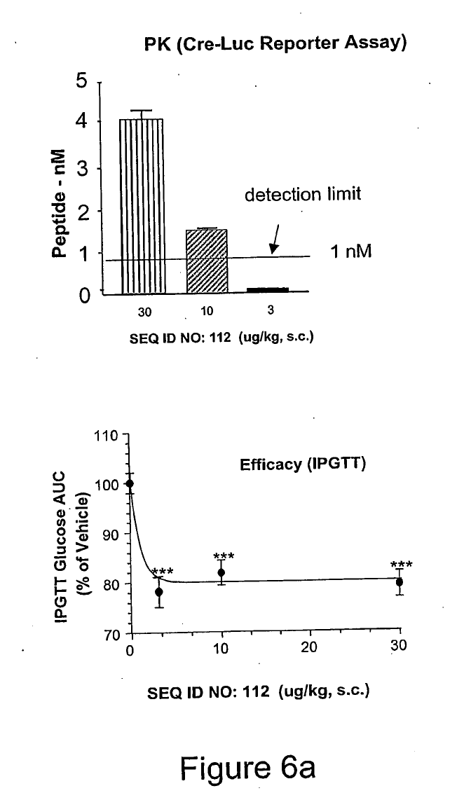

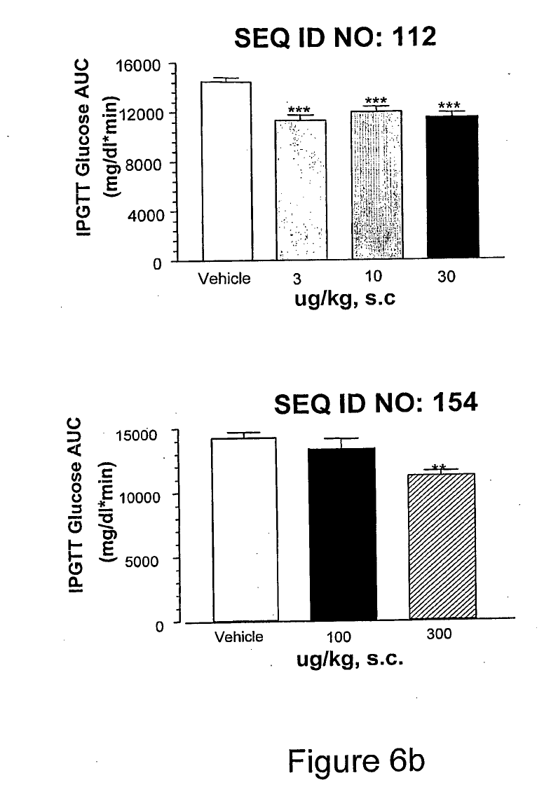

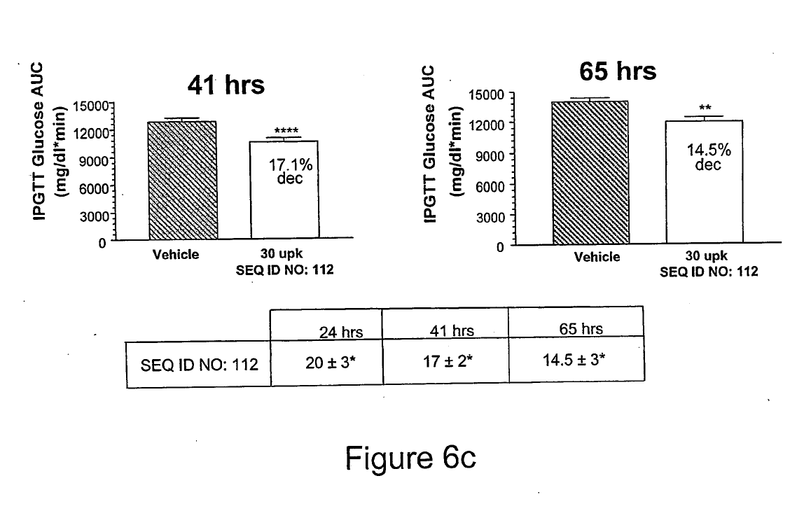

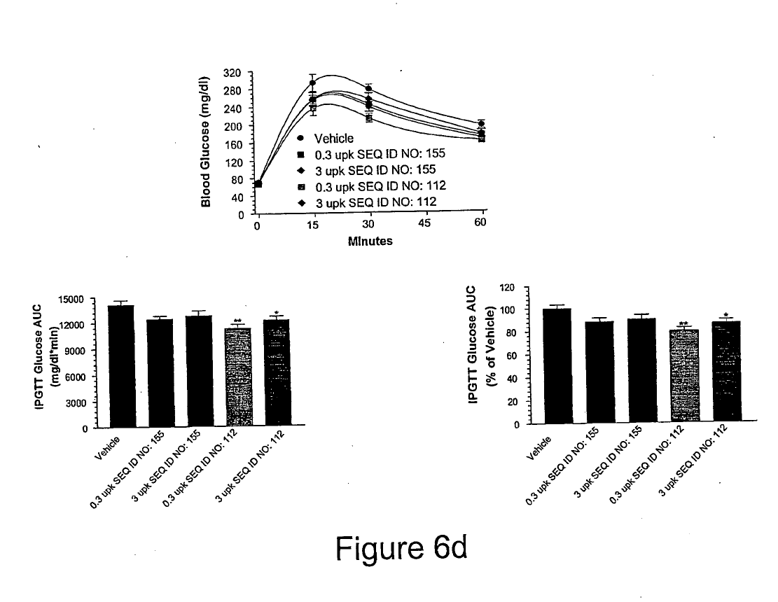

The in vivo activity of the PEGylated peptides of this invention when administered subcutaneously was examined in rats. Rats fasted overnight were given a subcutaneous injection of control or PEGylated peptide (1-100 μg/kg). Three hours later, basal blood glucose was measured, and the rats were given 2 g/kg of glucose intraperitoneally. Blood glucose was measured again after 15, 30, and 60 minutes. A PEGylated peptide of this invention significantly reduced blood glucose levels relative to the vehicle following the IPGTT (Intraperitoneal Glucose Tolerance Test), with 17%-28% reduction in the glucose AUC (FIGS. 6a-6d). This demonstrates that the PEGylated peptide has prolonged glucose lowering activity in vivo. In addition to the glucose lowering activity of the PEGylated peptides of the present invention, it also indicates prolonged peptide half-life in vivo. PACAP-27 has a very short half-life in vivo (<10 minutes). The ability of the PEGylated peptides of the invention to lower blood glucose at 24 (FIG. 6b), 41 (FIG. 6c), 65 (FIG. 6c), and 72 (FIG. 6d) hours following peptide administration is a clear indication that the peptide is present in the circulation at this time point and hence, has prolonged half-life relative to PACAP-27 (FIG. 6c). Furthermore, the peptides of SEQ ID NO: 112 demonstrates greater potency and efficacy at longer time points as compared to the peptides of SEQ ID NO: 154 and 155 (FIGS. 6b and 6d).

Demonstration of the activity of the polypeptides of the present invention may be accomplished through in vitro, ex vivo, and in vivo assays that are well known in the art. For example, to demonstrate the efficacy of a pharmaceutical agent for the treatment of diabetes and related disorders such as Syndrome X, impaired glucose tolerance, impaired fasting glucose, and hyperinsulinemia; atherosclerotic disease and related disorders such as hypertriglyceridemia and hypercholesteremia; and obesity, the following assays may be used.

### Method for Measuring Blood Glucose Levels

db/db mice (obtained from Jackson Laboratories, Bar Harbor, Me.) are bled (by either eye or tail vein) and grouped according to equivalent mean blood glucose levels. They are dosed orally (by gavage in a pharmaceutically acceptable vehicle) with the test polypeptide once daily for 14 days. At this point, the animals are bled again by eye or tail vein and blood glucose levels were determined. In each case, glucose levels are measured with a Glucometer Elite XL (Bayer Corporation, Elkhart, Ind.).

### Method for Measuring an Effect on Cardiovascular Parameters

Cardiovascular parameters (e.g., heart rate and blood pressure) are also evaluated. SHR rats are orally dosed once daily with vehicle or test polypeptide for 2 weeks. Blood pressure and heart rate are determined using a tail-cuff method as described by Grinsell, et al., (Am. J. Hypertens. 13:370-375, 2000). In monkeys, blood pressure and heart rate are monitored as described by Shen, et al., (J. Pharmacol. Exp. Therap. 278:1435-1443, 1996).

### Method for Measuring Triglyceride Levels

hApoA1 mice (obtained from Jackson Laboratories, Bar Harbor, Me.) are bled (by either eye or tail vein) and grouped according to equivalent mean serum triglyceride levels. They are dosed orally (by gavage in a pharmaceutically acceptable vehicle) with the test polypeptide once daily for 8 days. The animals are then bled again by eye or tail vein, and serum triglyceride levels are determined. In each case, triglyceride levels are measured using a Technicon Axon Autoanalyzer (Bayer Corporation, Tarrytown, N.Y.).

### Method for Measuring HDL-Cholesterol Levels

To determine plasma HDL-cholesteral levels, hApoA1 mice are bled and grouped with equivalent mean plasma HDL-cholesterol levels. The mice are orally dosed once daily with vehicle or test polypeptide for 7 days, and then bled again on day 8. Plasma is analyzed for HDL-cholesterol using the Synchron Clinical System (CX4) (Beckman Coulter, Fullerton, Calif.).

### Method for Measuring Total Cholesterol, HDL-Cholesterol, Triglycerides, and Glucose Levels

In another in vivo assay, obese monkeys are bled, then orally dosed once daily with vehicle or test polypeptide for 4 weeks, and then bled again. Serum is analyzed for total cholesterol, HDL-cholesterol, triglycerides, and glucose using the Synchron Clinical System (CX4) (Beckman Coulter, Fullerton, Calif.). Lipoprotein subclass analysis is performed by NMR spectroscopy as described by Oliver, et al., (Proc. Natl. Aced. Sci. USA 98:5306-5311, 2001).

All publications and patents mentioned in the above specification are incorporated herein by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein.

We claim:

1. A polypeptide selected from the group consisting of SEQ ID NOs: 1 to 148, and functionally equivalent fragments, derivatives, and variants thereof.

2. The polypeptide of claim 1, wherein said polypeptide is selected from the group consisting of SEQ ID NOs: 1, 2, 3, 4, 5, 112, 113, 114, 115, and 116.

3. An antibody which binds specifically to the polypeptide of claim 1.

4. The antibody of claim 3, wherein said antibody is a polyclonal antibody.

5. The antibody of claim 3, wherein said antibody is a monoclonal antibody.

6. An antibody which binds specifically to the polyethylene glycol.

7. The antibody of claim 6, wherein said antibody is a polyclonal antibody.

8. The antibody of claim 6, wherein said antibody is a monoclonal antibody.

9. A method for detecting a polypeptide selected from the group consisting of SEQ ID NOs: 1 to 148 in a sample comprising:
a. contacting the sample with an antibody of claim 3 or claim 6,
b. detecting said antibody, and
c. correlating the detection of antibody with the amount of polypeptide in the sample.

10. A method for detecting a polypeptide selected from the group consisting of SEQ ID NOs: 1 to 148 in a sample comprising:
a. contacting the sample with a first antibody of claim 3 or claim 6,
b. contacting the sample with a second labeled antibody, wherein the second antibody binds to the first antibody,
c. detecting the label, and
d. correlating the detection of label with the amount of polypeptide in the sample.

11. A kit for detecting a polypeptide selected from the group consisting of SEQ ID NOs: 1 to 148 in a sample comprising: a first antibody of claim 3 or claim 6 and a second antibody wherein the second antibody binds to the first antibody.

12. A pharmaceutical composition comprising a therapeutically effective amount of a polypeptide of claim 1, or functionally equivalent fragments, derivatives, and variants thereof, in combination with a pharmaceutically acceptable carrier.

13. The pharmaceutical composition of claim 12, wherein said polypeptide is selected from the group consisting of SEQ ID NOs: 1, 2, 3, 4, 5, 112, 113, 114, 115, and 116.

14. A pharmaceutical composition comprising a therapeutically effective amount of a polypeptide of claim 1, or functionally equivalent fragments, derivatives, and variants thereof, in combination with a pharmaceutically acceptable carrier and one or more pharmaceutical agents.

15. The pharmaceutical composition of claim 14, wherein said pharmaceutical agent is selected from the group consisting of PPAR ligands, insulin secretagogues, sulfonylurea drugs, α-glucosidase inhibitors, insulin sensitizers, hepatic glucose output lowering compounds, insulin and insulin derivatives, biguanides, protein tyrosine phosphatase-1B, dipeptidyl peptidase IV, 11 beta-HSD inhibitors, anti-obesity drugs, HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

16. A composition comprising an effective amount of a polypeptide of claim 1, or functionally equivalent fragments, derivatives, and variants thereof, in combination with an inert carrier.

17. A method of treating diabetes comprising the step of administering to a subject in need thereof a therapeutically effective amount of a polypeptide of claim 1 or a pharmaceutical composition of claim 12.

18. The method of claim 17, wherein said diabetes is selected from the group consisting of type 2 diabetes, maturity-onset diabetes of the young, latent autoimmune diabetes adult, and gestational diabetes.

19. A method of treating Syndrome X comprising the step of administering to a subject in need thereof a therapeutically effective amount of a polypeptide of claim 1 or a pharmaceutical composition of claim 12.

20. A method of treating diabetes-related disorders comprising the step of administering to a subject in need thereof a therapeutically effective amount of a polypeptide of claim 1 or a pharmaceutical composition of claim 12.

21. The method of claim 20, wherein said diabetes-related disorder is selected from the group consisting of hyperglycemia, hyperinsulinemia, impaired glucose tolerance, impaired fasting glucose, dyslipidemia, hypertriglycerldemia, and insulin resistance.

22. A method of treating diabetes comprising the step of administering to a subject in need thereof a therapeutically effective amount of a polypeptide of claim 1 in combination with one or more pharmaceutical agents.

23. The method of claim 20, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

24. The method of claim 23, wherein said diabetes is selected from the group consisting of type 2 diabetes, maturity-onset diabetes of the young, latent autoimmune diabetes adult, and gestational diabetes.

25. A method of treating Syndrome X comprising the step of administering to a subject in need thereof a therapeutically effective amount of a polypeptide of claim 1 in combination with one or more pharmaceutical agents.

26. The method of claim 25, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

27. A method of treating diabetes-related disorders comprising the step of administering to a subject in need thereof a therapeutically effective amount of a polypeptide of claim 1 in combination with one or more pharmaceutical agents.

28. The method of claim 27, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

29. The method of claim 28, wherein said pharmaceutical agent is selected from the group consisting of PPAR agonists, sulfonylurea drugs, non-sulfonylurea secretagogues, a glucosidase inhibitors, insulin sensitizers, insulin secretagogues, hepatic glucose output lowering compounds, insulin, and anti-obesity agents.

30. A method of treating diabetes, Syndrome X, or diabetes-related disorders comprising the step of administering to a subject in need thereof a therapeutically effective amount of a polypeptide of claim 1 in combination with one or more agents selected from the group consisting of HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

31. The method of claim 30, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

32. The method of any one of claims 22 to 31, wherein the polypeptide of claim 1 and one or more pharmaceutical agents are administered as a single pharmaceutical dosage formulation.

33. A method of treating or preventing secondary causes of diabetes comprising the step of administering to a subject in need thereof a therapeutically effective amount of a polypeptide of claim 1 or a pharmaceutical composition of claim 12.

34. The method of claim 33, wherein said secondary cause is selected from the group consisting of glucocorticoid excess, growth hormone excess, pheochromocytoma, and drug-induced diabetes.

35. A method of treating or preventing secondary causes of diabetes comprising the step of administering a subject in need thereof a therapeutically effective amount of a polypeptide of claim 1 in combination with one or more pharmaceutical agents.

36. The method of claim 35, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

37. A method of treating respiratory disease comprising the step of administering to a subject in need thereof a therapeutically effective amount of a polypeptide of claim 1 or a pharmaceutical composition of claim 12.

38. A method of treating obesity comprising the step of administering to a subject in need thereof a therapeutically effective amount of a polypeptide of claim 1 or a pharmaceutical composition of claim 12.

39. A method of treating cardiovascular disease comprising the step of administering to a subject in need thereof a therapeutically effective amount of a polypeptide of claim 1 or a pharmaceutical composition of claim 12.

40. The method of claim 39, wherein said cardiovascular disease is selected from atherosclerosis, coronary heart disease, coronary artery disease, and hypertension.

41. A method of treating disorders of lipid and carbohydrate metabolism comprising the step of administering to a subject in need thereof a therapeutically effective amount of a polypeptide of claim 1 or a pharmaceutical composition of claim 12.

42. A method of treating sleep disorders comprising the step of administering to a subject in need thereof a therapeutically effective amount of a polypeptide of claim 1 or a pharmaceutical composition of claim 12.

43. A method of treating male reproductive disorders comprising the step of administering to a subject in need thereof a therapeutically effective amount of a polypeptide of claim 1 or a pharmaceutical composition of claim 12.

44. A method of treating growth disorders or disorders of energy homeostasis comprising the step of administering to a subject in need thereof a therapeutically effective amount of a polypeptide of claim 1 or a pharmaceutical composition of claim 12.

45. A method of treating immune diseases comprising the step of administering to a subject in need thereof a therapeutically effective amount of a polypeptide of claim 1 or a pharmaceutical composition of claim 12.

46. A method of treating autoimmune diseases comprising the step of administering to a subject in need thereof a therapeutically effective amount of a polypeptide of claim 1 or a pharmaceutical composition of claim 12.

47. A method of treating acute and chronic inflammatory diseases comprising the step of administering to a subject in need thereof a therapeutically effective amount of a polypeptide of claim 1 or a pharmaceutical composition of claim 12.

48. A method of treating septic shock comprising the step of administering to a subject in need thereof a therapeutically effective amount of a polypeptide of claim 1 or a pharmaceutical composition of claim 12.

49. A method of stimulating insulin release in a glucose-dependent manner in a subject in need thereof by administering to said subject a polypeptide of claim 1 or a pharmaceutical composition of claim 12.

50. Polypeptides according to claim 1 for the treatment and/or prophylaxis of diabetes and diabetes-related disorders.

51. Medicament containing at least one polypeptide according to claim 1 in combination with at least one pharmaceutically acceptable, pharmaceutically safe carrier or excipient.

52. Use of polypeptides according to claim 1 for manufacturing a medicament for the treatment and/or prophylaxis of diabetes and diabetes-related disorders.

53. Medicament according to claim 51 for the treatment and/or prophylaxis of diabetes.


SEQUENCE LISTING (155 sequences)

SEQ ID NO: 1 - protein, 31 aa, Homo sapiens
  MOD_RES (1)..(31): ACETYLATION
HSDAVFTDQYTRLRKQVAAKKYLQSIKQKRY

SEQ ID NO: 2 - protein, 31 aa, Homo sapiens
  MOD_RES (1)..(31): ACETYLATION
HSDAVFTDQYTRLRKQMAAKKYLQSIKQKRY

SEQ ID NO: 3 - protein, 29 aa, Homo sapiens
  MOD_RES (1)..(29): ACETYLATION
HSDAVFTDQYTRLRKQVAAKKYLQSIKQK

SEQ ID NO: 4 - protein, 31 aa, Homo sapiens
  MOD_RES (1)..(31): ACETYLATION
HTEAVFTDQYTRLRKQVAAKKYLQSIKQKRY

SEQ ID NO: 5 - protein, 31 aa, Homo sapiens
  MOD_RES (1)..(31): ACETYLATION
HSDAVFTDQYTRLRKQLAVKKYLQDIKQGGT

SEQ ID NO: 6 - protein, 30 aa, Homo sapiens
  MOD_RES (1)..(30): ACETYLATION
HSDAVFTDQYTRLRKQMAAKKYLQSIKQKR

SEQ ID NO: 7 - protein, 31 aa, Homo sapiens
  MOD_RES (1)..(31): ACETYLATION
HSDAVFTDQYTRLRKQLAAKKYLQTIKQKRY

SEQ ID NO: 8 - protein, 31 aa, Homo sapiens
  MOD_RES (1)..(31): ACETYLATION
HSDAVFTDQYTRLRKQMAAKKYLQTIKQKRY

SEQ ID NO: 9 - protein, 31 aa, Homo sapiens
  MOD_RES (1)..(31): ACETYLATION
HSDAVFTDQYTRLRKQMAAHKYLQSIKQKRY

SEQ ID NO: 10 - protein, 31 aa, Homo sapiens
  MOD_RES (1)..(31): ACETYLATION
HSDAVFTDQYTRLRKQMAAKHYLQSIKQKRY

SEQ ID NO: 11 - protein, 30 aa, Homo sapiens
  MOD_RES (1)..(30): ACETYLATION
HSDAVFTDQYTRLRKQMAGKKYLQSIKQKR

SEQ ID NO: 12 - protein, 30 aa, Homo sapiens
  MOD_RES (1)..(30): ACETYLATION
HSDAVFTDQYTRLRKQMAKKKYLQSIKQKR

SEQ ID NO: 13 - protein, 30 aa, Homo sapiens
  MOD_RES (1)..(30): ACETYLATION
HSDAVFTDQYTRLRKQMARKKYLQSIKQKR

SEQ ID NO: 14 - protein, 30 aa, Homo sapiens
  MOD_RES (1)..(30): ACETYLATION
HSDAVFTDQYTRLRKQMASKKYLQSIKQKR

SEQ ID NO: 15 - protein, 30 aa, Homo sapiens
  MOD_RES (1)..(30): ACETYLATION
HSDAVFTDQYTRLRKQMAAKKYLQSIPQKR

SEQ ID NO: 16 - protein, 30 aa, Homo sapiens
  MOD_RES (1)..(30): ACETYLATION
HSDAVFTDQYTRLRKQMAAKKYLQSIQQKR

SEQ ID NO: 17 - protein, 30 aa, Homo sapiens
  MOD_RES (1)..(30): ACETYLATION
HSDAVFTDQYTRLRKQMAAKKYLQSIRQKR

SEQ ID NO: 18 - protein, 30 aa, Homo sapiens
  MOD_RES (1)..(30): ACETYLATION
HSDAVFTDQYTRLRKQMAAKKYLQSIKQRR

SEQ ID NO: 19 - protein, 30 aa, Homo sapiens
  MOD_RES (1)..(30): ACETYLATION
HSDAVFTDQYTRLRKQMAAKKYLQSIKQKA

SEQ ID NO: 20 - protein, 30 aa, Homo sapiens
  MOD_RES (1)..(30): ACETYLATION
HSDAVFTDQYTRLRKQMAAKKYLQSIKQKF

SEQ ID NO: 21 - protein, 30 aa, Homo sapiens
  MOD_RES (1)..(30): ACETYLATION
HSDAVFTDQYTRLRKQMAAKKYLQSIKQKH

SEQ ID NO: 22 - protein, 30 aa, Homo sapiens
  MOD_RES (1)..(30): ACETYLATION
HSDAVFTDQYTRLRKQMAAKKYLQSIKQKI

SEQ ID NO: 23 - protein, 30 aa, Homo sapiens
  MOD_RES (1)..(30): ACETYLATION
HSDAVFTDQYTRLRKQMAAKKYLQSIKQKK

SEQ ID NO: 24 - protein, 30 aa, Homo sapiens
  MOD_RES (1)..(30): ACETYLATION
HSDAVFTDQYTRLRKQMAAKKYLQSIKQKL

SEQ ID NO: 25 - protein, 30 aa, Homo sapiens
  MOD_RES (1)..(30): ACETYLATION
HSDAVFTDQYTRLRKQMAAKKYLQSIKQKM

SEQ ID NO: 26 - protein, 30 aa, Homo sapiens
  MOD_RES (1)..(30): ACETYLATION
HSDAVFTDQYTRLRKQMAAKKYLQSIKQKP

SEQ ID NO: 27 - protein, 30 aa, Homo sapiens
  MOD_RES (1)..(30): ACETYLATION
HSDAVFTDQYTRLRKQMAAKKYLQSIKQKQ

SEQ ID NO: 28 - protein, 30 aa, Homo sapiens
  MOD_RES (1)..(30): ACETYLATION
HSDAVFTDQYTRLRKQMAAKKYLQSIKQKS

SEQ ID NO: 29 - protein, 30 aa, Homo sapiens
  MOD_RES (1)..(30): ACETYLATION
HSDAVFTDQYTRLRKQMAAKKYLQSIKQKT

SEQ ID NO: 30 - protein, 30 aa, Homo sapiens
  MOD_RES (1)..(30): ACETYLATION
HSDAVFTDQYTRLRKQMAAKKYLQSIKQKV

SEQ ID NO: 31 - protein, 30 aa, Homo sapiens
  MOD_RES (1)..(30): ACETYLATION
HSDAVFTDQYTRLRKQMAAKKYLQSIKQKW

SEQ ID NO: 32 - protein, 30 aa, Homo sapiens
  MOD_RES (1)..(30): ACETYLATION
HSDAVFTDQYTRLRKQMAAKKYLQSIKQKY

SEQ ID NO: 33 - protein, 30 aa, Homo sapiens
  MOD_RES (1)..(30): ACETYLATION
HSDAVFTDQYTRLRKQMAGKKYLQSIKQRI

SEQ ID NO: 34 - protein, 30 aa, Homo sapiens
  MOD_RES (1)..(30): ACETYLATION
HSDAVFTDQYTRLRKQMAKKKYLQSIKQRI

SEQ ID NO: 35 - protein, 30 aa, Homo sapiens
  MOD_RES (1)..(30): ACETYLATION
HSDAVFTDQYTRLRKQMASKKYLQSIKQRI

SEQ ID NO: 36 - protein, 30 aa, Homo sapiens
  MOD_RES (1)..(30): ACETYLATION
HSDAVFTDQYTRLRKQMAAKKYLQSIPQRI

SEQ ID NO: 37 - protein, 30 aa, Homo sapiens
  MOD_RES (1)..(30): ACETYLATION
HSDAVFTDQYTRLRKQMASKKYLQSIRQRI

SEQ ID NO: 38 - protein, 31 aa, Homo sapiens
  MOD_RES (1)..(31): ACETYLATION
HSDAVFTDNYTRLRKQVAAKKYLQSIKQKRY

SEQ ID NO: 39 - protein, 31 aa, Homo sapiens
  MOD_RES (1)..(31): ACETYLATION
HSDAVFTDNYTRLRKQMAAKKYLQSIKQKRY

SEQ ID NO: 40 - protein, 29 aa, Homo sapiens
  MOD_RES (1)..(29): ACETYLATION
HSDAVFTDNYTRLRKQVAAKKYLQSIKQK

SEQ ID NO: 41 - protein, 31 aa, Homo sapiens
  MOD_RES (1)..(31): ACETYLATION
HTEAVFTDNYTRLRKQVAAKKYLQSIKQKRY

SEQ ID NO: 42 - protein, 31 aa, Homo sapiens
  MOD_RES (1)..(31): ACETYLATION
HSDAVFTDNYTRLRKQLAVKKYLQDIKQGGT

SEQ ID NO: 43 - protein, 30 aa, Homo sapiens
  MOD_RES (1)..(30): ACETYLATION
HSDAVFTDNYTRLRKQMAAKKYLQSIKQKR

SEQ ID NO: 44 - protein, 31 aa, Homo sapiens
  MOD_RES (1)..(31): ACETYLATION
HSDAVFTDNYTRLRKQLAAKKYLQTIKQKRY

SEQ ID NO: 45 - protein, 31 aa, Homo sapiens
  MOD_RES (1)..(31): ACETYLATION
HSDAVFTDNYTRLRKQMAAKKYLQTIKQKRY

SEQ ID NO: 46 - protein, 31 aa, Homo sapiens
  MOD_RES (1)..(31): ACETYLATION
HSDAVFTDNYTRLRKQMAAHKYLQSIKQKRY

SEQ ID NO: 47 - protein, 31 aa, Homo sapiens
  MOD_RES (1)..(31): ACETYLATION
HSDAVFTDNYTRLRKQMAAKHYLQSIKQKRY

SEQ ID NO: 48 - protein, 30 aa, Homo sapiens
  MOD_RES (1)..(30): ACETYLATION
HSDAVFTDNYTRLRKQMAGKKYLQSIKQKR

SEQ ID NO: 49 - protein, 30 aa, Homo sapiens
  MOD_RES (1)..(30): ACETYLATION
HSDAVFTDNYTRLRKQMAKKKYLQSIKQKR

SEQ ID NO: 50 - protein, 30 aa, Homo sapiens
  MOD_RES (1)..(30): ACETYLATION
HSDAVFTDNYTRLRKQMARKKYLQSIKQKR

SEQ ID NO: 51 - protein, 30 aa, Homo sapiens
  MOD_RES (1)..(30): ACETYLATION
HSDAVFTDNYTRLRKQMASKKYLQSIKQKR

SEQ ID NO: 52 - protein, 30 aa, Homo sapiens
  MOD_RES (1)..(30): ACETYLATION
HSDAVFTDNYTRLRKQMAAKKYLQSIPQKR

SEQ ID NO: 53 - protein, 30 aa, Homo sapiens
  MOD_RES (1)..(30): ACETYLATION
HSDAVFTDNYTRLRKQMAAKKYLQSIQQKR

SEQ ID NO: 54 - protein, 30 aa, Homo sapiens
  MOD_RES (1)..(30): ACETYLATION
HSDAVFTDNYTRLRKQMAAKKYLQSIRQKR

SEQ ID NO: 55 - protein, 30 aa, Homo sapiens
  MOD_RES (1)..(30): ACETYLATION
HSDAVFTDNYTRLRKQMAAKKYLQSIKQRR

SEQ ID NO: 56 - protein, 30 aa, Homo sapiens
  MOD_RES (1)..(30): ACETYLATION
HSDAVFTDNYTRLRKQMAAKKYLQSIKQKA

SEQ ID NO: 57 - protein, 30 aa, Homo sapiens
  MOD_RES (1)..(30): ACETYLATION
HSDAVFTDNYTRLRKQMAAKKYLQSIKQKF

SEQ ID NO: 58 - protein, 30 aa, Homo sapiens
  MOD_RES (1)..(30): ACETYLATION
HSDAVFTDNYTRLRKQMAAKKYLQSIKQKH

SEQ ID NO: 59 - protein, 30 aa, Homo sapiens
  MOD_RES (1)..(30): ACETYLATION
HSDAVFTDNYTRLRKQMAAKKYLQSIKQKI

SEQ ID NO: 60 - protein, 30 aa, Homo sapiens
  MOD_RES (1)..(30): ACETYLATION
HSDAVFTDNYTRLRKQMAAKKYLQSIKQKK

SEQ ID NO: 61 - protein, 30 aa, Homo sapiens
  MOD_RES (1)..(30): ACETYLATION
HSDAVFTDNYTRLRKQMAAKKYLQSIKQKL

SEQ ID NO: 62 - protein, 30 aa, Homo sapiens
  MOD_RES (1)..(30): ACETYLATION
HSDAVFTDNYTRLRKQMAAKKYLQSIKQKM

SEQ ID NO: 63 - protein, 30 aa, Homo sapiens
  MOD_RES (1)..(30): ACETYLATION
HSDAVFTDNYTRLRKQMAAKKYLQSIKQKP

SEQ ID NO: 64 - protein, 30 aa, Homo sapiens
  MOD_RES (1)..(30): ACETYLATION
HSDAVFTDNYTRLRKQMAAKKYLQSIKQKQ

SEQ ID NO: 65 - protein, 30 aa, Homo sapiens
  MOD_RES (1)..(30): ACETYLATION
HSDAVFTDNYTRLRKQMAAKKYLQSIKQKS

SEQ ID NO: 66 - protein, 30 aa, Homo sapiens
  MOD_RES (1)..(30): ACETYLATION
HSDAVFTDNYTRLRKQMAAKKYLQSIKQKT

SEQ ID NO: 67 - protein, 30 aa, Homo sapiens
  MOD_RES (1)..(30): ACETYLATION
HSDAVFTDNYTRLRKQMAAKKYLQSIKQKV

SEQ ID NO: 68 - protein, 30 aa, Homo sapiens
  MOD_RES (1)..(30): ACETYLATION
HSDAVFTDNYTRLRKQMAAKKYLQSIKQKW

SEQ ID NO: 69 - protein, 30 aa, Homo sapiens
  MOD_RES (1)..(30): ACETYLATION
HSDAVFTDNYTRLRKQMAAKKYLQSIKQKY

SEQ ID NO: 70 - protein, 30 aa, Homo sapiens
  MOD_RES (1)..(30): ACETYLATION
HSDAVFTDNYTRLRKQMAGKKYLQSIKQRI

SEQ ID NO: 71 - protein, 30 aa, Homo sapiens
  MOD_RES (1)..(30): ACETYLATION
HSDAVFTDNYTRLRKQMAKKKYLQSIKQRI

SEQ ID NO: 72 - protein, 30 aa, Homo sapiens
  MOD_RES (1)..(30): ACETYLATION
HSDAVFTDNYTRLRKQMASKKYLQSIKQRI

SEQ ID NO: 73 - protein, 30 aa, Homo sapiens
  MOD_RES (1)..(30): ACETYLATION
HSDAVFTDNYTRLRKQMAAKKYLQSIPQRI

SEQ ID NO: 74 - protein, 30 aa, Homo sapiens
  MOD_RES (1)..(30): ACETYLATION
HSDAVFTDNYTRLRKQMASKKYLQSIRQRI

SEQ ID NO: 75 - protein, 31 aa, Homo sapiens
  MOD_RES (1)..(31): ACETYLATION
HSDAVFTDQYTRLRKQVAAKKYLQSIKNKRY

SEQ ID NO: 76 - protein, 31 aa, Homo sapiens
  MOD_RES (1)..(31): ACETYLATION
HSDAVFTDQYTRLRKQMAAKKYLQSIKNKRY

SEQ ID NO: 77 - protein, 29 aa, Homo sapiens
  MOD_RES (1)..(29): ACETYLATION
HSDAVFTDQYTRLRKQVAAKKYLQSIKNK

SEQ ID NO: 78 - protein, 31 aa, Homo sapiens
  MOD_RES (1)..(31): ACETYLATION
HTEAVFTDQYTRLRKQVAAKKYLQSIKNKRY

SEQ ID NO: 79 - protein, 31 aa, Homo sapiens
  MOD_RES (1)..(31): ACETYLATION
HSDAVFTDQYTRLRKQLAVKKYLQDIKNGGT

SEQ ID NO: 80 - protein, 30 aa, Homo sapiens
  MOD_RES (1)..(30): ACETYLATION
HSDAVFTDQYTRLRKQMAAKKYLQSIKNKR

SEQ ID NO: 81 - protein, 31 aa, Homo sapiens
  MOD_RES (1)..(31): ACETYLATION
HSDAVFTDQYTRLRKQLAAKKYLQTIKNKRY

SEQ ID NO: 82 - protein, 31 aa, Homo sapiens
  MOD_RES (1)..(31): ACETYLATION
HSDAVFTDQYTRLRKQMAAKKYLQTIKNKRY

SEQ ID NO: 83 - protein, 31 aa, Homo sapiens
  MOD_RES (1)..(31): ACETYLATION
HSDAVFTDQYTRLRKQMAAHKYLQSIKNKRY

SEQ ID NO: 84 - protein, 31 aa, Homo sapiens
  MOD_RES (1)..(31): ACETYLATION
HSDAVFTDQYTRLRKQMAAKHYLQSIKNKRY

SEQ ID NO: 85 - protein, 30 aa, Homo sapiens
  MOD_RES (1)..(30): ACETYLATION
HSDAVFTDQYTRLRKQMAGKKYLQSIKNKR

SEQ ID NO: 86 - protein, 30 aa, Homo sapiens
  MOD_RES (1)..(30): ACETYLATION
HSDAVFTDQYTRLRKQMAKKKYLQSIKNKR

SEQ ID NO: 87 - protein, 30 aa, Homo sapiens
  MOD_RES (1)..(30): ACETYLATION
HSDAVFTDQYTRLRKQMARKKYLQSIKNKR

SEQ ID NO: 88 - protein, 30 aa, Homo sapiens
  MOD_RES (1)..(30): ACETYLATION
HSDAVFTDQYTRLRKQMASKKYLQSIKNKR

SEQ ID NO: 89 - protein, 30 aa, Homo sapiens
  MOD_RES (1)..(30): ACETYLATION
HSDAVFTDQYTRLRKQMAAKKYLQSIPNKR

SEQ ID NO: 90 - protein, 30 aa, Homo sapiens
  MOD_RES (1)..(30): ACETYLATION
HSDAVFTDQYTRLRKQMAAKKYLQSIQNKR

SEQ ID NO: 91 - protein, 30 aa, Homo sapiens
  MOD_RES (1)..(30): ACETYLATION
HSDAVFTDQYTRLRKQMAAKKYLQSIRNKR

SEQ ID NO: 92 - protein, 30 aa, Homo sapiens
  MOD_RES (1)..(30): ACETYLATION
HSDAVFTDQYTRLRKQMAAKKYLQSIKNRR

SEQ ID NO: 93 - protein, 30 aa, Homo sapiens
  MOD_RES (1)..(30): ACETYLATION
HSDAVFTDQYTRLRKQMAAKKYLQSIKNKA

SEQ ID NO: 94 - protein, 30 aa, Homo sapiens
  MOD_RES (1)..(30): ACETYLATION
HSDAVFTDQYTRLRKQMAAKKYLQSIKNKF

SEQ ID NO: 95 - protein, 30 aa, Homo sapiens
  MOD_RES (1)..(30): ACETYLATION
HSDAVFTDQYTRLRKQMAAKKYLQSIKNKH

SEQ ID NO: 96 - protein, 30 aa, Homo sapiens
  MOD_RES (1)..(30): ACETYLATION
HSDAVFTDQYTRLRKQMAAKKYLQSIKNKI

SEQ ID NO: 97 - protein, 30 aa, Homo sapiens
  MOD_RES (1)..(30): ACETYLATION
HSDAVFTDQYTRLRKQMAAKKYLQSIKNKK

SEQ ID NO: 98 - protein, 30 aa, Homo sapiens
  MOD_RES (1)..(30): ACETYLATION
HSDAVFTDQYTRLRKQMAAKKYLQSIKNKL

SEQ ID NO: 99 - protein, 30 aa, Homo sapiens
  MOD_RES (1)..(30): ACETYLATION
HSDAVFTDQYTRLRKQMAAKKYLQSIKNKM

SEQ ID NO: 100 - protein, 30 aa, Homo sapiens
  MOD_RES (1)..(30): ACETYLATION
HSDAVFTDQYTRLRKQMAAKKYLQSIKNKP

SEQ ID NO: 101 - protein, 30 aa, Homo sapiens
  MOD_RES (1)..(30): ACETYLATION
HSDAVFTDQYTRLRKQMAAKKYLQSIKNKQ

SEQ ID NO: 102 - protein, 30 aa, Homo sapiens
  MOD_RES (1)..(30): ACETYLATION
HSDAVFTDQYTRLRKQMAAKKYLQSIKNKS

SEQ ID NO: 103 - protein, 30 aa, Homo sapiens
  MOD_RES (1)..(30): ACETYLATION
HSDAVFTDQYTRLRKQMAAKKYLQSIKNKT

SEQ ID NO: 104 - protein, 30 aa, Homo sapiens
  MOD_RES (1)..(30): ACETYLATION
HSDAVFTDQYTRLRKQMAAKKYLQSIKNKV

SEQ ID NO: 105 - protein, 30 aa, Homo sapiens
  MOD_RES (1)..(30): ACETYLATION
HSDAVFTDQYTRLRKQMAAKKYLQSIKNKW

SEQ ID NO: 106 - protein, 30 aa, Homo sapiens
  MOD_RES (1)..(30): ACETYLATION
HSDAVFTDQYTRLRKQMAAKKYLQSIKNKY

SEQ ID NO: 107 - protein, 30 aa, Homo sapiens
  MOD_RES (1)..(30): ACETYLATION
HSDAVFTDQYTRLRKQMAGKKYLQSIKNRI

SEQ ID NO: 108 - protein, 30 aa, Homo sapiens
  MOD_RES (1)..(30): ACETYLATION
HSDAVFTDQYTRLRKQMAKKKYLQSIKNRI

SEQ ID NO: 109 - protein, 30 aa, Homo sapiens
  MOD_RES (1)..(30): ACETYLATION
HSDAVFTDQYTRLRKQMASKKYLQSIKNRI

SEQ ID NO: 110 - protein, 30 aa, Homo sapiens
  MOD_RES (1)..(30): ACETYLATION
HSDAVFTDQYTRLRKQMAAKKYLQSIPNRI

SEQ ID NO: 111 - protein, 30 aa, Homo sapiens
  MOD_RES (1)..(30): ACETYLATION
HSDAVFTDQYTRLRKQMASKKYLQSIRNRI

SEQ ID NO: 112 - protein, 32 aa, Homo sapiens
  MISC_FEATURE (1)..(32): Cysteine at position 32 is PEGylated.
HSDAVFTDQYTRLRKQVAAKKYLQSIKQKRYC

SEQ ID NO: 113 - protein, 32 aa, Homo sapiens
  MISC_FEATURE (1)..(32): Cysteine at position 32 is PEGylated.
HSDAVFTDQYTRLRKQMAAKKYLQSIKQKRYC

SEQ ID NO: 114 - protein, 30 aa, Homo sapiens
  MISC_FEATURE (1)..(30): Cysteine at position 30 is PEGylated.
HSDAVFTDQYTRLRKQVAAKKYLQSIKQKC

SEQ ID NO: 115 - protein, 32 aa, Homo sapiens
  MISC_FEATURE (1)..(32): Cysteine at position 32 is PEGylated.
HTEAVFTDQYTRLRKQVAAKKYLQSIKQKRYC

SEQ ID NO: 116 - protein, 32 aa, Homo sapiens
  MISC_FEATURE (1)..(32): Cysteine at position 32 is PEGylated.
HSDAVFTDQYTRLRKQLAVKKYLQDIKQGGTC

SEQ ID NO: 117 - protein, 31 aa, Homo sapiens
  MISC_FEATURE (1)..(31): Cysteine at position 31 is PEGylated.
HSDAVFTDQYTRLRKQMAAKKYLQSIKQKRC

SEQ ID NO: 118 - protein, 32 aa, Homo sapiens
  MISC_FEATURE (1)..(32): Cysteine at position 32 is PEGylated.
HSDAVFTDQYTRLRKQLAAKKYLQTIKQKRYC

SEQ ID NO: 119 - protein, 32 aa, Homo sapiens
  MISC_FEATURE (1)..(32): Cysteine at position 32 is PEGylated.
HSDAVFTDQYTRLRKQMAAKKYLQTIKQKRYC

SEQ ID NO: 120 - protein, 32 aa, Homo sapiens
  MISC_FEATURE (1)..(32): Cysteine at position 32 is PEGylated.
HSDAVFTDQYTRLRKQMAAHKYLQSIKQKRYC

SEQ ID NO: 121 - protein, 32 aa, Homo sapiens
  MISC_FEATURE (1)..(32): Cysteine at position 32 is PEGylated.
HSDAVFTDQYTRLRKQMAAKHYLQSIKQKRYC

SEQ ID NO: 122 - protein, 31 aa, Homo sapiens
  MISC_FEATURE (1)..(31): Cysteine at position 31 is PEGylated.
HSDAVFTDQYTRLRKQMAGKKYLQSIKQKRC

SEQ ID NO: 123 - protein, 31 aa, Homo sapiens
  MISC_FEATURE (1)..(31): Cysteine at position 31 is PEGylated.
HSDAVFTDQYTRLRKQMAKKKYLQSIKQKRC

SEQ ID NO: 124 - protein, 31 aa, Homo sapiens
  MISC_FEATURE (1)..(31): Cysteine at position 31 is PEGylated.
HSDAVFTDQYTRLRKQMARKKYLQSIKQKRC

SEQ ID NO: 125 - protein, 31 aa, Homo sapiens
  MISC_FEATURE (1)..(31): Cysteine at position 31 is PEGylated.
HSDAVFTDQYTRLRKQMASKKYLQSIKQKRC

SEQ ID NO: 126 - protein, 31 aa, Homo sapiens
  MISC_FEATURE (1)..(31): Cysteine at position 31 is PEGylated.
HSDAVFTDQYTRLRKQMAAKKYLQSIPQKRC

SEQ ID NO: 127 - protein, 31 aa, Homo sapiens
  MISC_FEATURE (1)..(31): Cysteine at position 31 is PEGylated.
HSDAVFTDQYTRLRKQMAAKKYLQSIQQKRC

SEQ ID NO: 128 - protein, 31 aa, Homo sapiens
  MISC_FEATURE (1)..(31): Cysteine at position 31 is PEGylated.
HSDAVFTDQYTRLRKQMAAKKYLQSIRQKRC

SEQ ID NO: 129 - protein, 31 aa, Homo sapiens
  MISC_FEATURE (1)..(31): Cysteine at position 31 is PEGylated.
HSDAVFTDQYTRLRKQMAAKKYLQSIKQRRC

SEQ ID NO: 130 - protein, 31 aa, Homo sapiens
  MISC_FEATURE (1)..(31): Cysteine at position 31 is PEGylated.
HSDAVFTDQYTRLRKQMAAKKYLQSIKQKAC

SEQ ID NO: 131 - protein, 31 aa, Homo sapiens
  MISC_FEATURE (1)..(31): Cysteine at position 31 is PEGylated.
HSDAVFTDQYTRLRKQMAAKKYLQSIKQKFC

SEQ ID NO: 132 - protein, 31 aa, Homo sapiens
  MISC_FEATURE (1)..(31): Cysteine at position 31 is PEGylated.
HSDAVFTDQYTRLRKQMAAKKYLQSIKQKHC

SEQ ID NO: 133 - protein, 31 aa, Homo sapiens
  MISC_FEATURE (1)..(31): Cysteine at position 31 is PEGylated.
HSDAVFTDQYTRLRKQMAAKKYLQSIKQKIC

SEQ ID NO: 134 - protein, 31 aa, Homo sapiens
  MISC_FEATURE (1)..(31): Cysteine at position 31 is PEGylated.
HSDAVFTDQYTRLRKQMAAKKYLQSIKQKKC

SEQ ID NO: 135 - protein, 31 aa, Homo sapiens
  MISC_FEATURE (1)..(31): Cysteine at position 31 is PEGylated.
HSDAVFTDQYTRLRKQMAAKKYLQSIKQKLC

SEQ ID NO: 136 - protein, 31 aa, Homo sapiens
  MISC_FEATURE (1)..(31): Cysteine at position 31 is PEGylated.
HSDAVFTDQYTRLRKQMAAKKYLQSIKQKMC

SEQ ID NO: 137 - protein, 31 aa, Homo sapiens
  MISC_FEATURE (1)..(31): Cysteine at position 31 is PEGylated.
HSDAVFTDQYTRLRKQMAAKKYLQSIKQKPC

SEQ ID NO: 138 - protein, 31 aa, Homo sapiens
  MISC_FEATURE (1)..(31): Cysteine at position 31 is PEGylated.
HSDAVFTDQYTRLRKQMAAKKYLQSIKQKQC

SEQ ID NO: 139 - protein, 31 aa, Homo sapiens
  MISC_FEATURE (1)..(31): Cysteine at position 31 is PEGylated.
HSDAVFTDQYTRLRKQMAAKKYLQSIKQKSC

SEQ ID NO: 140 - protein, 31 aa, Homo sapiens
  MISC_FEATURE (1)..(31): Cysteine at position 31 is PEGylated.
HSDAVFTDQYTRLRKQMAAKKYLQSIKQKTC

SEQ ID NO: 141 - protein, 31 aa, Homo sapiens
  MISC_FEATURE (1)..(31): Cysteine at position 31 is PEGylated.
HSDAVFTDQYTRLRKQMAAKKYLQSIKQKVC

SEQ ID NO: 142 - protein, 31 aa, Homo sapiens
  MISC_FEATURE (1)..(31): Cysteine at position 31 is PEGylated.
HSDAVFTDQYTRLRKQMAAKKYLQSIKQKWC

SEQ ID NO: 143 - protein, 31 aa, Homo sapiens
  MISC_FEATURE (1)..(31): Cysteine at position 31 is PEGylated.
HSDAVFTDQYTRLRKQMAAKKYLQSIKQKYC

SEQ ID NO: 144 - protein, 31 aa, Homo sapiens
  MISC_FEATURE (1)..(31): Cysteine at position 31 is PEGylated.
HSDAVFTDQYTRLRKQMAGKKYLQSIKQRIC

SEQ ID NO: 145 - protein, 31 aa, Homo sapiens
  MISC_FEATURE (1)..(31): Cysteine at position 31 is PEGylated.
HSDAVFTDQYTRLRKQMAKKKYLQSIKQRIC

SEQ ID NO: 146 - protein, 31 aa, Homo sapiens
  MISC_FEATURE (1)..(31): Cysteine at position 31 is PEGylated.
HSDAVFTDQYTRLRKQMASKKYLQSIKQRIC

SEQ ID NO: 147 - protein, 31 aa, Homo sapiens
  MISC_FEATURE (1)..(31): Cysteine at position 31 is PEGylated.
HSDAVFTDQYTRLRKQMAAKKYLQSIPQRIC

SEQ ID NO: 148 - protein, 31 aa, Homo sapiens
  MISC_FEATURE (1)..(31): Cysteine at position 31 is PEGylated.
HSDAVFTDQYTRLRKQMASKKYLQSIRQRIC

SEQ ID NO: 149 - protein, 38 aa, Homo sapiens
HSDGIFTDSYSRYRKQMAVKKYLAAVLGKRYKQRVKNK

SEQ ID NO: 150 - protein, 27 aa, Homo sapiens
HSDGIFTDSYSRYRKQMAVKKYLAAVL

SEQ ID NO: 151 - protein, 28 aa, Homo sapiens
HSDAVFTDNYTRLRKQMAVKKYLNSILN

SEQ ID NO: 152 - protein, 31 aa, Homo sapiens; identical to SEQ ID NO: 1

SEQ ID NO: 153 - protein, 31 aa, Homo sapiens
  MOD_RES (1)..(31): ACETYLATION
HTDAVFTDQYTRLRKQVAAKKYLQSIKQKRY

SEQ ID NO: 154 - protein, 32 aa, Homo sapiens; identical to SEQ ID NO: 112
  MISC_FEATURE (1)..(32): Cysteine at position 32 is PEGylated.

SEQ ID NO: 155 - protein, 32 aa, Homo sapiens
  MOD_RES (1)..(32): ACETYLATION
HTDAVFTDQYTRLRKQVAAKKYLQSIKQKRYC